(19) United States
(12) Patent Application Publication
(10) Pub. No.: US 2025/0319183 A1
(43) Pub. Date: Oct. 16, 2025

(54) COMPOSITIONS AND METHODS FOR THE TREATMENT OF CANCER

(71) Applicants: David AVIGAN, Sharon, MA (US); Jacalyn ROSENBLATT, Newton, MA (US); Dina STROOPINSKY, Newton, MA (US); Beth Israel Deaconess Medical Center, BOSTON, MA (US); Dana-Farber Cancer Institute, Inc., BOSTON, MA (US)

(72) Inventors: DAVID AVIGAN, SHARON, MA (US); JACALYN ROSENBLATT, NEWTON, MA (US); DONALD KUFE, WELLESLEY, MA (US); DINA STROOPINSKY, NEWTON, MA (US)

(21) Appl. No.: 18/554,429

(22) PCT Filed: Apr. 7, 2022
(86) PCT No.: PCT/US2022/023849

§ 371 Date: Oct. 10, 2023

Related U.S. Application Data
(60) Provisional application No. 63/171,808, filed on Apr. 7, 2021.

(51) Int. Cl. A61K 40/19; A61K 31/454; A61K 31/635; A61K 31/706; A61K 39/00; A61K 40/24; A61P 35/02

(52) U.S. Cl. CPC A61K 40/19; A61K 31/454; A61K 31/635; A61K 31/706; A61K 40/24; A61P 35/02; A61K 2039/804; A61K 2239/48

(57) ABSTRACT

The present invention relates, in part, to the use of dendritic cell-tumor fusion vaccines and a Bcl-2 inhibitor (e.g., venetoclax) to treat or prevent certain cancers.

### RELATED APPLICATIONS

This application claims the benefit of U.S. Provisional Application No. 63/171,808, filed Apr. 7, 2021, which is hereby incorporated by reference in its entirety.

### STATEMENT OF RIGHTS

This invention was made with government support under grant number R01 CA212649 awarded by the National Institutes of Health. The government has certain rights in the invention.

### BACKGROUND OF THE INVENTION

There are more than 100 distinct forms of cancer that are generally characterized by the unregulated proliferation of abnormal cancerous cells. The large number of distinct cancer forms along with the particular stages that describe disease progression and distinct subject profiles requires an expansive pharmacopeia for effective treatment. Subject tolerance of treatment and drug resistant cancers complicate, and in some cases eliminate, treatment options. For example, elderly subjects and/or unfit subjects may not tolerate aggressive therapies (e.g., chemotherapy), leading to cessation of therapy and adoption of an alternative therapy that may have a lower success rate of reducing disease progression or attaining remission. Drug resistance is a principal factor in the inability to cure some cancers and the resulting development of multi-agent therapies (Vasan et al. (2019) Nature 575:299-309). Another complicating factor is that immunotherapies are impeded by the immunosuppressive milieu in the tumor microenvironment can impair antigen presentation, increase regulatory T-cells, dysregulate checkpoint pathways, and increase the burden of myeloid-derived suppressor cells (MDSCs) (Andersen (2014) Leukemia 28:1784-1792; Isidori et al. (2014) Expert Review of Hematology 7:807-818; Schlöβer et al. (2014) Immunotherapy 6:973-988; Sharma & Allison (2015) Science 348:56-61).

Acute Myeloid Leukemia (AML) is an example of a lethal hematological malignancy for which chemotherapy is rarely curative. Treatment options are often impacted by subject condition (e.g., age) and clonal resistance to particular therapies. Elderly AML subjects respond to standard therapies at lower rates than younger subjects (40%-60% of elderly subjects achieve complete remission compared to 60%-80% of younger subjects) (Zhang et al. (2019) Onco. Targets Ther. 12:1937-1945). Approximately 60% of elderly subjects undergoing first-line chemotherapy suffered a recurrence and >85% of subjects failed in treatment (primary resistance and relapse after induction therapy) (Döhner et al. (2010) Blood 115(3):453-474; Bryan et al. (2015) Drugs Aging. 32(8):623-637). Resistance to treatment can result from AML cells having genetic alterations, signaling pathway errors, immunosuppression, and/or overexpressing one of more proteins associated with resistance (e.g., multidrug resistance-related protein (MRP1), P-glycoprotein (P-gp), glutathione S-transferase (GST), topoisomerase II, and protein kinase C).

Recently, decitabine, a hypomethylation agent (HMA), and venetoclax, a Bcl-2 inhibitor, have been adopted as a standard of care combination therapy administered to subjects with AML, and this regimen is well tolerated by elderly subjects (DiNardo et al. (Blood) 133(1):7-17). However, less than 40% of the subjects achieved complete remission, and remission duration was slightly less than one year. Subjects that respond positively to any treatment and achieve complete remission are routinely assessed for minimum/measured residual disease (MRD) to detect the presence of leukemia cells. MRD is highly prognostic of long-term outcome, as subjects who exceed the MRD threshold (e.g., at 1:104 to 1:106 blast: white blood cell) are considered at higher risk of relapse (Schuurhuis et al. (2018) Blood 131(12):1275-1291). Residual AML cells that are drug resistant or that have a poor-risk cytogenetic profile can become the predominant clone in a recurrence, making a second complete remission less likely.

Accordingly, additional therapeutic interventions are necessary for preventing and treating cancers like AML, including cancers in subjects characterized as having MRD.

### SUMMARY OF THE INVENTION

The present invention is based, at least in part, on the discovery that administration of a dendritic cell (DC)-tumor fusion vaccine rescues negative immunomodulatory effects to cancer expected from administration of a Bcl-2 inhibitor (e.g., venetoclax), either alone or in combination with a hypomethylating agent (HMA). Without being bound by theory, Bcl-2 inhibitors, such as venetoclax, have an unwanted side effect of inducing lymphopenia and suppressing blood count levels. Accordingly, immunotherapies are expected to be ineffective given the suppression of immune cells (e.g., myelosuppression) and to disrupt function of the DC-tumor fusion vaccine cells in presenting antigens and activating T cells. However, it is described herein that the treatment and/or prevention of cancer may be unexpectedly enhanced by administering to a subject in need a Bcl-2 inhibitor, such as venetoclax, along with an immunotherapy that is a DC-tumor fusion vaccine.

Counterintuitively, the DC-tumor fusion vaccine provides effective immune responses despite immunosuppressive effects of Bcl-2 inhibitors, such as venetoclax. In in vitro studies, DC/tumor fusion vaccine stimulation of autologous T cells in the presence of HMA and venetoclax resulted in enhanced expansion of CD4 and CD8 T cells expressing IFNg, increased T cell activation measured by CD25/69 expression, and increased expansion of antigen specific T cells measured by CD137 expression as compared to that observed with vaccine alone. By contrast, the combination of DC-tumor fusion vaccination and Bcl-2 inhibitor (e.g., venetoclax) with HMA therapy was unexpectedly not associated with increased CD8 T cell expression of PD-1. A dose relationship was observed and this could be blunted by dose escalation of the HMA. In in vivo experiments, mice challenged with the murine TIB-49 AML cell line and then treated with the combination of a DC-tumor fusion vaccination and Bcl-2 inhibitor (e.g., venetoclax) with HMA therapy showed better survival as compared to those treated with venetoclax and HMA, or vaccination, alone. For example, animals receiving venetoclax and HMA showed a decreased presence of tumor specific T cells that was effectively expanded by combination with the vaccine as manifested by the percentage of cells expressing interferon gamma (IFNγ) in response to ex vivo exposure tumor lysate exposure. The combination of vaccination with venetoclax (and HMA) resulted in the expansion of T cells targeting the tumor antigen, survivin. Thus, the combination of DC-tumor fusion vaccination and Bcl-2 inhibitor (e.g., venetoclax) provides synergy with respect to T cell activation and the generation of tumor specific immunity in vitro and in vivo. The Bcl-2 inhibitor may be administered in combination with a hypomethylating agent (HMA) as part of a standard of care therapy for a cancer (e.g., acute myeloid leukemia (AML)). For example, administration of a DC-tumor fusion vaccine in conjunction with a Bcl-2 inhibitor therapy results in increased activated T cells activity (e.g., IFN-γ expression) relative to the Bcl-2 inhibitor alone. Accordingly, DC-tumor fusion vaccines are useful in enhancing immune cell function in a subject having a cancer treated with a Bcl-2 inhibitors, and this combination therapy represents a novel strategy for treating cancer.

Accordingly, one aspect of the presentation invention provides a method of treating a cancer in a subject, the method comprising administering to the subject therapeutically effective amounts of a dendritic cell (DC)/tumor fusion vaccine and a Bcl-2 inhibitor.

Another aspect of the present invention provides a method of preventing a recurrence of a cancer in a subject, the method comprising administering to the subject therapeutically effective amounts of a DC/tumor fusion vaccine and a Bcl-2 inhibitor.

In yet another embodiment, a method is provided for prolonging survival of a subject having or suspected of having a cancer, the method comprising administering to the subject therapeutically effective amounts of a DC/tumor fusion vaccine and a Bcl-2 inhibitor.

Numerous embodiments are further provided that can be applied to any aspect of the present invention and/or combined with any other embodiment described herein. For example, in one embodiment, the methods further comprise administering to the subject a hypomethylation agent, optionally wherein the hypomethylation agent is further administered as a maintenance treatment. In another embodiment, the cancer is a solid tumor or hematological cancer. In still another embodiment, the hematological cancer is acute myeloid leukemia (AML), multiple myeloma (MM), or chronic lymphocytic leukemia (CLL). In yet another embodiment, the Bcl-2 inhibitor is a BH3 mimetic. In one embodiment, the Bcl-2 mimetic is venetoclax. In another embodiment, the hypomethylation agent is decitabine, 5-azacytidine, guadecitabine, or 5-fluro-2′-deoxycytidine. In still another embodiment, the DC/tumor fusion vaccine is autogenic. In yet another embodiment, the DC/tumor fusion vaccine is allogenic.

In another embodiment, the methods of the present invention further comprise administering to the subject an immune checkpoint inhibitor. In one embodiment, the checkpoint inhibitor is selected from the group of a PD-1 inhibitor, a TIM-3 inhibitor, LAG3 inhibitor, TIGIT inhibitor, B7H3 inhibitor, CD39 inhibitor, CD73 inhibitor and adenosine A2A receptor. In yet another embodiment, the checkpoint inhibitor is an antibody that specifically binds to a checkpoint peptide.

In still another embodiment, the methods of the present invention further comprise administering to the subject an immunomodulatory agent. In one embodiment, the immunomodulatory agent is lenalidomide or pomalinomide.

In yet another embodiment, the methods of the present invention further comprise administering to the subject a cytokine. In one embodiment, the cytokine is granulocyte-macrophage colony-stimulating factor (GM-CSF).

In another embodiment, the methods of the present invention further comprise administering an IDO inhibitor.

In still another embodiment, the methods of the present invention further comprise a toll-like receptor (TLR) agonist, CpG oligodeoxynucleotides (CPG-ODNs), polyinosinic-polycytidylic acid (polyIC), or tetanus toxoid.

In another embodiment, the methods of the present invention further comprise administering a MUC1 inhibitor. In one embodiment, the MUC1 inhibitor is GO-203.

In yet another embodiment, the methods of the present invention further comprise administering to the subject a therapeutically effective amount of a MUC16 inhibitor.

In another embodiment, the subject is in remission. In still another embodiment, the subject has minimal residual disease.

### BRIEF DESCRIPTION OF THE DRAWINGS

The patent of application file contains at least one drawing executed in color. Copies of this patent or patent application publication with color drawing(s) will be provided by the office upon request and payment of the necessary fee.

FIG. 1 shows a diagram of a dendritic cell (DC)/tumor hybrid cell formed by the fusion of an antigen presenting DC to an acute myeloid leukemia or multiple myeloma cell, wherein the dendritic cell (DC)/tumor hybrid cell expresses or otherwise comprises a cancer antigen and other costimulatory peptides.

FIG. 2A-FIG. 2C show that vaccination with a DC-tumor fusion in conjunction with decitabine and venetoclax results in decreased mortality and increased T cell activity in subjects relative to subjects that received only the DC-tumor fusion vaccine or decitabine and venetoclax alone. FIG. 2A is a series of images comparing subjects at day 14 and day 30, which shows that fewer subjects that received both the DC-fusion vaccine and decitabine and venetoclax died relative to the subjects that received either the C-tumor fusion vaccine or decitabine and venetoclax. FIG. 2B shows the upregulation of activity in tumor-specific T cells relative to T cells specific for a non-tumor antigen (i.e., cytomegalovirus (CMV) antigen. The upregulation of activity was most pronounced in subjects that received a DC-tumor vaccine in conjunction with decitabine and venetoclax. FIG. 2C shows an upregulation of IFN-γ expression in T cells in subjects that received a DC-tumor vaccine in conjunction with decitabine and venetoclax relative to subjects that received either a DC-tumor vaccine or decitabine and venetoclax.

FIG. 3A-3C show that hypomethylation augments tumor antigen presentation. FIG. 3A are images of cells treated with and without SGI-110, a hypomethylating agent. FIG. 3B is a graph showing the mean fluorescent intensity observed in THP-1 cells expressing PR1, an HLA-A2-restricted leukemia-associated peptide/antigen in AML. The terms “us” denotes unstained and “NT” denotes no treatment. FIG. 3C shows the percentages of CD4 and CD8 cells expressing IFNγ after treatment with a DC/tumor fusion vaccine, SGI-110, or a DC/tumor fusion vaccine and SGI-110. FIG. 3B and FIG. 3C show results from peripheral blood analysis at day 21.

FIG. 4A-FIG. 4E show synergy between vaccination with DC/AML fusion cells and checkpoint inhibition. FIG. 4A is a timeline depicting the relevant administration and analysis time points. “TIB-49” refers to a murine myeloid leukemia cell line isolated from a C57BL/6J mouse. “BLI” refers to bioluminescence imaging. FIG. 4B shows the clonal proportions observed after DC/AML vaccination and checkpoint inhibitor treatment, DC/AML vaccination only, and no treatment. FIG. 4C are PET scans of mice treated with IgG, checkpoint inhibitors, a DC/AML fusion vaccine, and a DC/AML fusion vaccine combined with checkpoint inhibitors. FIG. 4D shows the overall survival of mice treated with IgG, checkpoint inhibitors, a DC/AML fusion vaccine, and a DC/AML fusion vaccine combined with checkpoint inhibitors. FIG. 4E shows the survival of mice after rechallenge with TIB-49 cells.

FIG. 5 shows a representative, non-limiting in vivo treatment schema with fusion vaccine and decitabine+venetoclax. C57BL/6J mice were inoculated retro-orbitally with 150×10³ luciferase/mCherry TIB-49 murine leukemia cells. Cohorts of mice were treated with fusion vaccine alone, decitabine+venetoclax, or combination of fusion vaccine and decitabine+venetoclax. Mice were treated with decitabine 0.4 mg/kg on days 7-9 as well as venetoclax 100 mg/kg on days 7-11. Mice were treated with DC/AML fusion cells via subcutaneous injection on Day 14. They were subsequently treated with venetoclax 100 mg/kg on days 14-18 and days 21-25.

FIG. 6 shows effects of venetoclax on cytotoxic T lymphocyte (CTL) mediated killing of leukemia (Tib-49) cells in vitro. Leukemia cells were treated with venetoclax at doses 1 μM and 5 uM for 48 hours. Following treatment, cells were co-cultured with vaccine educated T-cells (Tf) in an effector:target ratio of 10:1. Cytotoxicity was assessed using the Promega BrightGlo® Luciferase Assay System. Each condition was normalised to untreated tumor and tumor treated with venetoclax 1 μM and 5 uM, respectively, without incubation with Tf. Venetoclax treated tumor cells were more prone to CTL-mediated killing than untreated tumor.

FIG. 7 shows results of dynamic BH3 profiling. AML cells were exposed to venetoclax for 16 hours or DMSO followed by BH3 profiling with BIM peptides. Sensitivity to BH3 peptides was measured as cytochrome c release.

FIG. 8 shows cytochrome c release (%) by AML cells exposed to 1 nM venetoclax and 3 nM venetoclax, after co-culture with autologous non-vaccine educated T cells and vaccine-educated T cells. Increased cytochrome c release was observed following exposure to increasing doses of venetoclax and co-culture of vaccine educated T cells compared to non-vaccine educated T cells.

### DETAILED DESCRIPTION OF THE INVENTION

It has been determined herein that administering a dendritic cell (DC)-tumor fusion vaccine to a subject receiving a Bcl-2-based therapy to treat a cancer rescues the immune response to the cancer that is muted during Bcl-2 inhibitor therapy. For example, administering a DC-tumor fusion vaccine in conjunction with decitabine and venetoclax results in increased immune cell activity relative to administration of either the DC-tumor fusion vaccine or the Bcl-2 inhibitor alone. Venetoclax has been shown to repress blood cell count levels, thus it is surprising that an immune response is generated after administration of the DC-tumor fusion vaccine and a Bcl-2 inhibitor. The DC-tumor fusion vaccine described herein combined with the anti-cancer activities of a Bcl-2 inhibitor, either alone or in combination with other anti-cancer agents, such as a hypomethylating agent, represents a novel approach to cancer therapy.

Accordingly, the present invention relates to methods for treating cancer with a DC-tumor fusion vaccine and a Bcl-2 inhibitor. Other aspects of the present invention relate to methods of preventing reoccurrence of a cancer or prolonging survival of a subject by administering a DC-tumor fusion vaccine and a Bcl-2 inhibitor. In some embodiments, the Bcl-2 inhibitor is venetoclax, and the subject also is administered a hypomethylation agent (HMA), such as decitabine. A combination of decitabine and venetoclax is a standard treatment for AML.

In order that the present invention may be more readily understood, certain terms are first defined. Additional definitions are set forth throughout the detailed description.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a subject suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CHI domains; (ii) a F(ab′) 2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, biomarker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′) 2 fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

By contrast, antigen-binding portions can be adapted to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “BCL2 Apoptosis Regulator (Bcl-2)” refers to an integral outer mitochondrial membrane protein that inhibits apoptosis in certain cells (e.g., lymphocytes). Bcl-2 binds to BCL2 Associated X, Apoptosis Regulator (BAX) and BCL2 Antagonist/Killer 1 (BAK), both of which are important cell death regulator proteins. BAX and BAC, when activated, create pores in the outer mitochondrion membrane thereby depolarizing the mitochondria, which releases caspases and other proteins that destroy the cell (Cory et al. (2002) Nat. Rev. Cancer 2(9):647-656; Letai (2008) Nat. Rev. Cancer 8(2):121-132). Thus, Bcl-2 acts as an apoptosis inhibitor, contributing to the survival and longevity of cancer cells.

A “Bcl-2 inhibitor” refers to any small molecule, protein (including but not limited to antibodies), nucleic acid (Schlagbauer-Wadl et al. (2000) J. Invest. Dermatol. 114(4):725-730; Ramanarayanan et al. (2004) Br. J. Haematol. 127(5):519-530), or any other molecule or composition that binds to at least one of Bcl-2, Bcl-XL, and Bcl-w, and antagonizes the activity of the Bcl-2 family related nucleic acid or protein, for example, inhibiting Bcl-2 from binding or otherwise activating BAX and BAK. Members of the BH3 protein family, which include BIM, PUMA, NOXA, BAD, HRK, BMF, and BIK, are naturally occurring Bcl-2 antagonists (Roberts (2020) Hematology 1:1-9). Of this family, BID, PUMA, BID, and BAD bind to Bcl-2. A balance between the proapoptotic proteins of the BH3 protein family and anti-apoptotic proteins such as Bcl-2 must be maintained for the proper functioning, and eventual death, of a cell. However, in some hematological cancers this balance is disrupted, and Bcl-2 may be upregulated, causes the cancer cells to be somewhat resistant to apoptosis. Bcl-2 inhibitors can be used to mitigate the effects of upregulated Bcl-2. Examples of Bcl-2 inhibitors include BGB-11417, G3139, oblimersen, BH3 mimetics, and inhibitors disclosed in U.S. Pat. Nos. 8,691,184; 10,829,488; 9,872,861, the contents of each are incorporated herein in their entirety, and the like.

The nucleic acid and amino acid sequences of representative human BCL-2 isoforms are available to the public at the GenBank database and is shown in Table 1. Human BCL-2 isoforms, generated by alternative splicing, include the longer isoform alpha precursor (GenBank database numbers NP_000624.2 and NP_000633.3) and the shorter isoform beta (NM_000648.2 and NP_000657.3). The BCL-2 gene is conserved in at least chimpanzee, dog, cow, mouse, rat, chicken, and frog. Nucleic acid and polypeptide sequences of BCL-2 orthologs in organisms other than humans are well-known and include, for example, chimpanzee (Pan troglodytes) BCL-2 (XM 001145537.4→XP 001145537.1; XM_016933838.2→XP_016789327.1; XM 016933839.2→XP_016789328.1), dog BCL-2 (NM_001002949.1→NP_001002949.1), cattle BCL-2 (NM 001166486.1→NP 001159958.1, XM 024984174.1→XP 024839942.1, XM 005224105.4→XP 005224162.1, XM 024984176.1→XP_024839944.1, and XM 024984175.1→XP 024839943.1), mouse BCL-2 (NM_009741.5→NP 033871.2 and NM 177410.3→NP_803129.2), rat (Rattus norvegicus) BCL-2 (NM_016993.2→NP 058689.2 and XR_005492200.1), chicken (Gallus gallus) BCL-2 (NM_205339.2→NP_990670.2), and frog (XM_002934396.5→XP_002934442.1).

The term “BCL-2 activity” includes the ability of a BCL-2 polypeptide to bind to BAX and BAK, and the ability to promote apoptosis.

The term “BH3 mimetics” refers to a class of small molecule Bcl-2 inhibitors and is a representative class of Bcl-2 inhibitors. Examples of BH3 mimetics include ABT-737 (Abbott Laboratories, North Chicago, IL), which is considered the first-in-class BH3 mimetic, navitoclax and venetoclax (Abbott Laboratories, North Chicago, IL), gossypol compounds, and obatoclax (GX15-070) (Cournoyer et al. (2019) BMC Cancer 19:1018).

ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide; CAS No. 852808-04-9) has the following structure:

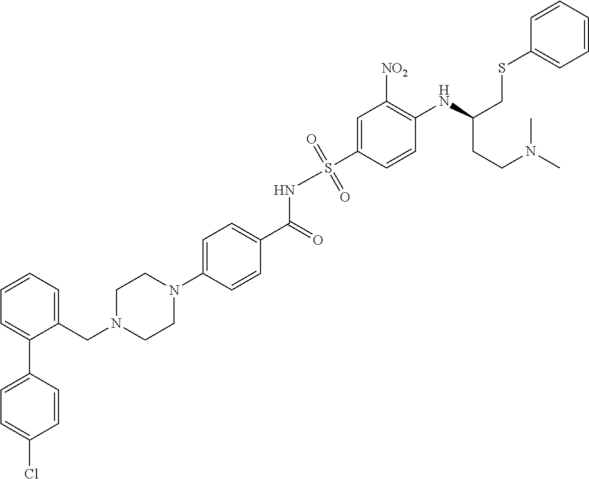 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

ABT-737 can bind to and block the function of Bcl-2 (Petros et al. (2006) J. Med. Chem. 49(2):656-63). It has shown single agent activity against multiple myeloma (MM) and AML cell lines (Chauhan et al. (2007) Oncogene 26(16):2374-80; Konopleva et al. (2006) Cancer Cell 10(5):375-88) as well as the ability to potentiate other anticancer therapies in AML, MM and chronic myelogenous leukemia (CML) (Oltersdorf et al. (2005) Nature 435(7042):677-81; Chauhan et al.; Konopleva et al.; Kuroda et al. (2006) Proc. Natl. Acad. Sci. USA 103(40):14907-12; Tse et al. (2008) Cancer Res. 68(9):3421-8. ABT-737 is less orally available than later subsequent derivatives of this molecule.

Navitoclax (4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide; CAS No. 923564-51-6) is a second generation BH3 mimetic and an orally available derivative of ABT-737 having the following structure:

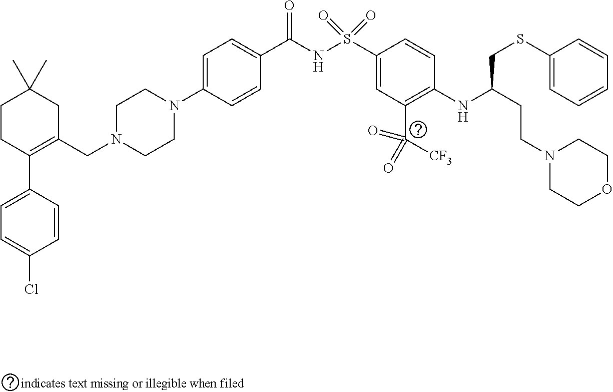

(Tse et al. (2008) Cancer Res. 68(9):3421-8; U.S. application Ser. No. 11/999,330 (US2008019987) and Ser. No. 12/005,688 (US20080269067), the contents of each are incorporated herein in their entirety). Having improved oral bioavailability profile, navitoclax has been shown to potentiate the response of B-cell lymphoma and MM cells to various standard anticancer therapies (Ackler et al. (2010) Cancer Chemother. Pharmacol. 66(5):869-80. This drug has shown anti-cancer activity in chronic lymphocytic leukemia (CLL) and AML cells (Roberts et al. (2012) J. Clin. Oncol. 30(5):488-96), small cell lung cancer (Faber et al. (2015) PNAS 112(11):E1288-96, and in combination with MEK1/2 inhibitor pimasertib (MEKI), ABT-263 efficiently targeted AML cells (Ariau et al. (2016) Oncogene 7(1):845-59.

Venetoclax (4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide; CAS No. 1257044-40-8) is another derivative of ABT-737 generated by reverse engineering of navitoclax to increase Bcl-2 selectivity (Souers et al. (2013) Nat. Med. 19(2):202-208) and has the following structure:

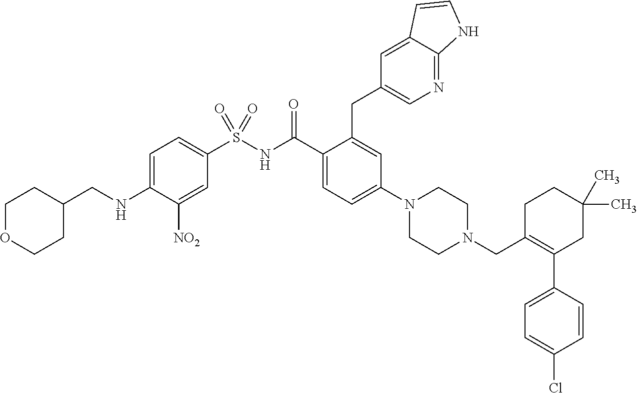 [SMILES: CC1(C)CCC(CN2CCN(c3ccc(C(=O)NS(=O)(=O)c4ccc(NCC5CCOCC5)c([N+](=O)[O-])c4)c(Cc4cnc5[nH]ccc5c4)c3)CC2)=C(c2ccc(Cl)cc2)C1]

(U.S. Pat. Nos. 8,546,399; 8,722,657; 9,174,982; 9,539,251; and 10,730,873). Venetoclax is a highly potent, orally bioavailable and BCL-2-selective inhibitor that can be used in combination with other anti-cancer agents. In xenograft models of hematological tumors, venetoclax inhibited tumor growth in a dose-dependent manner (Souers et al. 2013). Venetoclax has shown promise as a single agent in lymphoid malignancies and has demonstrated strong activity in AML (Lin et al. (2016) J. Clin. Oncol. 34:7007-7007; Stilgenbauer et al. (2016) Lancet Oncol. 17:768-778; Wei et al. (2016) Blood 128:102-102; Wei et al. (2017) Blood 130 (Suppl. 1):890-890). In human studies, venetoclax administration, generally in combination with another anti-cancer therapy, resulted in higher progression free survival rates in CLL (Seymour et al. (2018) N. Engl. J. Med.; 378:1107-1120), AML (Pollyea et al. (2019) Blood Adv. 3(24):4326-4335), small lymphocytic lymphoma (Seymour et al. (2017) Lancet Oncol. 18(2):230-240), and there are over 70 ongoing or completed clinical trials involving venetoclax for a host of distinct indications (e.g., multiple myeloma, AML, breast cancer, non-Hodgkin's lymphoma, mantle cell lymphoma, large B-cell, diffuse lymphoma, follicular lymphoma, and high grade B-cell lymphoma) (see the World Wide Web at clinicaltrials.gov, last visited Mar. 29, 2021). Anti-cancer therapeutics that can be combined with venetoclax to treat a cancer include, but are not limited to, cytarabine, cobimetinib, atezolizumab, rituximab, bendamustine, ibrutinib, obinutuzumab, ketoconazole, ixazomib citrate, dexamethasone, idelalisib, cobimetinib, idasanutlin, azatidine, idasanutlin, daratumumab, dexamethasone, bortezomib, polatuzumab, vedotin, prednisone, revlimid, etoposide, vincristine, cyclophosphamide, and doxorubicin.

Obatoclax ((2Z)-2-[(5Z)-5-[(3,5-dimethyl-1H-pyrrol-2-yl)methylidene]-4-methoxypyrrol-2-ylidene]indole, CAS No. 803712-67-6) is another small molecule inhibitor of Bcl-2 and has the following structure:

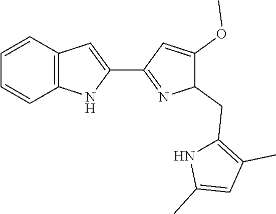 [SMILES: COC1=CC(c2cc3ccccc3[nH]2)=NC1Cc1[nH]c(C)cc1C]

Obatoclax treatment has been shown to Induce apoptosis and autophagy-dependent cell death of B cell Non-Hodgkin's lymphoma (B-NHL) cells (Hernandez-Ilizaliturri et al. (2008) Blood 112(11):605) and inhibits Bcl-2 binding to Bax and Bak resulting in enhanced TRAIL-mediated apoptosis (Huang and Sinicrope (Cancer Res. (2008) 68(9): 2390).

Gossypol compounds, such as gossypol and apogossypol are orally active compounds that behave as do the BH3 proteins. Removal of the two aldehyde groups in gossypol results in apogossypol. These and additional gossypol compounds are described in U.S. Pat. No. 8,163,805, the contents of which are incorporated herein in their entirety. Cell death induction by gossypol and apogossypol appears to be caspase-independent and may be only partially due to Bcl-2 inhibition (Volger et al. (2009) Cell Death Differentiation 16:1030-1039).

The term “biomarker” refers to a measurable entity of the present invention that has been determined to be informative of the therapeutic effects on a cancer or associated with a cancer. Biomarkers can include, without limitation, nucleic acids and proteins, including those described herein. As described herein, any relevant characteristic of a biomarker can be used, such as the copy number, amount, activity, location, modification (e.g., phosphorylation), and the like.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias. Cancer cells are often in the form of a tumor, but such cells may exist alone within an animal, or may be a non-tumorigenic cancer cell, such as a leukemia cell. As used herein, the term “cancer” includes premalignant as well as malignant cancers. “Tumor” can refer to solid tumors or a hematologic malignancy. Solid tumors can include, but are not limited to, breast or renal cancer tumors. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated. In certain embodiments, the cancer encompasses hematological cancers, such as acute myeloid leukemia (AML) and multiple myeloma.

As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents. Any two agents described herein can be administered conjointly, for example, a dendritic cell (DC)/tumor fusion vaccine and a Bcl-2 inhibitor can be administered conjointly according to the methods disclosed herein.

The term “hematological cancer” refers to cancers of cells derived from the blood. In some embodiments, the hematological cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), Hodgkin's lymphoma, mantle cell lymphoma (MCL), follicular lymphoma, Waldenstrom's macroglobulinemia (WM), B-cell lymphoma and diffuse large B-cell lymphoma (DLBCL). NHL may include indolent Non-Hodgkin's Lymphoma (iNHL) or aggressive Non-Hodgkin's Lymphoma (aNHL).

“Immunomodulatory agent” refers to any agent that can modulate an immune response in a subject. Non-limiting examples of immunomodulatory agents include lenalidomide, pomalinomide, and apremilast.

The term “leukemia” refers to a group of diseases that are cancers of the marrow and blood, where the malignant cells are white blood cells (leukocytes). The two major groups are lymphatic, and myeloid leukemia. Both groups are considered as either acute or chronic depending on various factors. Also included are lymphoid leukemias. Leukemias can thus be divided into four main types: acute lymphocytic leukemia, acute myelogenous leukemia, chronic lymphocytic leukemia and chronic myelogenous leukemia. Acute and chronic leukemias are usually studied as groups separated by the cells which are affected. These heterogeneous groups are usually considered together and are considered as a group of diseases characterized by infiltration of the bone marrow and other tissues by the cells of the hematopoietic system. The infiltration is called neoplastic, meaning new growth of cells, but all of the cells seen in the marrow, and peripheral circulation in leukemia are normal in a normal bone marrow, except for one structure, seen in myelocytic leukemia called Auer rods. These structures are repeated in this kind of leukemia, and are unknown as to structure, and relationship to any other material. Acute lymphoblastic leukemia (ALL) is also referred to as acute lymphocytic leukemia and acute lymphoid leukemia and is a form of leukemia characterized by excess lymphoblasts. Malignant, immature white blood cells continuously multiply and are overproduced in the bone marrow. ALL causes damage and death by crowding out normal cells in the bone marrow, and by spreading (infiltrating) to other organs. ALL is most common in childhood with a peak incidence at 2-5 years of age, and another peak in old age. Standard of care for treating ALL focuses on treatment of different phases in order to control bone marrow and systemic (whole-body) disease as well as to prevent leukemic cells from spreading to other sites, particularly the central nervous system (CNS), e.g., monthly lumbar punctures: a) induction chemotherapy is used to bring about bone marrow remission. For adults, standard induction plans include prednisone, vincristine, and an anthracycline drug; other drug plans may include L-asparaginase or cyclophosphamide. For children with low-risk ALL, standard therapy usually consists of three drugs (prednisone, L-asparaginase, and vincristine) for the first month of treatment; b) consolidation therapy or intensification therapy eliminates any remaining leukemia cells. There are many different approaches to consolidation, but it is typically a high-dose, multi-drug treatment that is undertaken for a few months. Subjects with low- to average-risk ALL receive therapy with antimetabolite drugs such as methotrexate and 6-mercaptopurine (6-MP). High-risk subjects receive higher drug doses of these drugs, plus additional drugs; c) CNS prophylaxis (preventive therapy) stops the cancer from spreading to the brain and nervous system in high-risk subjects. Standard prophylaxis may include radiation of the head and/or drugs delivered directly into the spine; and/or d) maintenance treatments with chemotherapeutic drugs prevent disease recurrence once remission has been achieved. Maintenance therapy usually involves lower drug doses, and may continue for up to three years. Alternatively, allogeneic bone marrow transplantation may be appropriate for high-risk or relapsed subjects. Chronic lymphocytic leukemia (also known as “chronic lymphoid leukemia” or “CLL”), is a leukemia of the white blood cells (lymphocytes) that affects a particular lymphocyte, the B cell, which originates in the bone marrow, develops in the lymph nodes, and normally fights infection. In CLL, the DNA of a B cell is damaged, so that it cannot fight infection, but grows out of control and crowds out the healthy blood cells that can fight infection. CLL is an abnormal neoplastic proliferation of B cells. The cells accumulate mainly in the bone marrow and blood. Although not originally appreciated, CLL is now thought to be identical to a disease called small lymphocytic lymphoma (SLL), a type of non-Hodgkin's lymphoma which presents primarily in the lymph nodes. Most people are diagnosed without symptoms as the result of a routine blood test that returns a high white blood cell count, but as it advances, CLL results in swollen lymph nodes, spleen, and liver, and eventually anemia and infections. Early CLL is not usually treated, and late CLL is treated with chemotherapy and monoclonal antibodies. Survival varies from 5 years to more than 25 years. It is now possible to diagnose subjects with short and long survival more precisely by examining the DNA mutations, and subjects with slowly-progressing disease can be reassured and may not need any treatment in their lifetimes [Chiorazzi et al., (2005) N. Engl. J. Med. 352(8):804-815]. Chronic myelogenous leukemia (CML), also known as chronic granulocytic leukemia (CGL), is a neoplastic disorder of the hematopoietic stem cell. In its early phases, this disease is characterized by leukocytosis, the presence of increased numbers of immature granulocytes in the peripheral blood, splenomegaly and anemia. These immature granulocytes include basophils, eosinophils, and neutrophils. The immature granulocytes also accumulate in the bone marrow, spleen, liver, and occasionally in other tissues. Subjects presenting with this disease characteristically have more than 75,000 white blood cells per microliter, and the count may exceed 500,000/μl. Cytologically, CML is characterized by a translocation between chromosome 22 and chromosome 9. This translocation juxtaposes a purported proto-oncogene with tyrosine kinase activity, a circumstance that apparently leads to uncontrolled cell growth. The resulting translocated chromosome is sometimes referred to as the Philadelphia chromosome.

The term “acute myeloid leukemia” as used herein, refers to a blood cancer that originates in the marrow and immature blood cells fail to mature resulting in an increased prevalence of blasts in the blood. The median age of AML subjects is 68 years. To diagnose AML, peripheral blood smears are performed to detect blasts and are compared to the complete blood count in a sample. Additionally genetic and cytogenetic tests are often performed to detect molecular features that may contribute to the severity of the disease or susceptibility or resistance to certain treatments. For example, certain mutations and chromosomal abnormalities are associated with increased risk (Kantarjian et al. (2021) Blood Cancer J. 11(2):41). Multiple recurrent somatic mutations in >90% of subjects with AML, including are FLT3, NPM1, DNMT3A, IDH1, IDH2, TET2, RUNX1, p53, NRAS, CEBPA, WT1 (Papaemmanuil et al. (2016) N. Engl. J. Med. 374, 2209-2221; Richard-Carpentier and DiNardo (2019) Hematol. Am. Soc. Hematol. Educ. Program 548-556; Angenendt, L. et al. J. Clin. Oncol. 37, 2632-2642). Additionally, approximately 5-10% of AML cases are acute promyelocytic leukemia, which is characterized by the cytogenetic abnormality t (15; 17). This abnormality results in the PML-RAR alpha fusion oncogene (Kantajarin et al. (2021).

For AML subjects in complete remission, residual disease is measured as part of the standard of care in AML (Jongen-Lavrencic et al. (2018) N. Engl. J. Med. 378, 1189-1199). Measurable residual disease in subjects in complete remission suggests a higher risk of relapse and worse prognosis. Thus, treatment decisions are based, at least partially, on the underlying genetic or cytogenic abnormalities and/or the presence of measured residual disease. As described herein, combination therapy comprising venetoclax and decitabine has become a standard of care treatment for AML subjects.

The term “lymphocytes” refers to cells of the immune system which are a type of white blood cell. Lymphocytes include, but are not limited to, T-cells (cytotoxic and helper T-cells), B-cells and natural killer cells (NK cells).

The term “lymphoma” refers to cancers that originate in the lymphatic system. Lymphoma is characterized by malignant neoplasms of lymphocytes-B lymphocytes and T lymphocytes (i.e., B-cells and T-cells). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body. Lymphomas include, but are not limited to, Hodgkin's lymphoma, non-Hodgkin's lymphoma, cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma and mantle zone lymphoma and low grade follicular lymphoma.

The term “multiple myeloma (MM)” refers an incurable hematological malignancy characterized by the accumulation of abnormal plasma cells in the bone marrow that impedes production of normal blood cells. The average survival of MM subjects has improved in recent years as a result of the introduction of proteasome inhibitors and immunomodulatory agents into treatment regimens but is still quite poor at only 5 years.

The term “coding region” refers to regions of a nucleotide sequence comprising codons that are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to an experimental variable. For example, a control may provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer subject (can be stored sample or previous sample measurement) with a known outcome; normal tissue or cells isolated from a subject, such as a normal subject or the cancer subject, cultured primary cells/tissues isolated from a subject such as a normal subject or the cancer subject, adjacent normal cells/tissues obtained from the same organ or body location of the cancer subject, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of subjects, or a set of subjects with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of subjects, such as a set of cancer subjects, or for a set of cancer subjects receiving a certain treatment, or for a set of subjects with one outcome versus another outcome. In the former case, the specific expression product level of each subject can be assigned to a percentile level of expression, or expressed as either higher or lower than the mean or average of the reference standard expression level. In another preferred embodiment, the control may comprise normal cells, cells from subjects treated with combination chemotherapy, and cells from subjects having benign cancer. In another embodiment, the control may also comprise a measured value for example, average level of expression of a particular gene in a population compared to the level of expression of a housekeeping gene in the same population. Such a population may comprise normal subjects, cancer subjects who have not undergone any treatment (i.e., treatment naive), cancer subjects undergoing standard of care therapy, or subjects having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of subject samples, such as all subjects with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control subjects with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. Immune cells that have received a cell-receptor mediated signal, e.g., via an activating receptor are referred to herein as “activated immune cells.”

The term “cytokine” refers to any of the numerous factors that exert a variety of effects on cells, for example, inducing growth or proliferation. Non-limiting examples of cytokines include, IL-2, stem cell factor (SCF), IL-3, IL-6, IL-7, IL-12, IL-15, G-CSF, GM-CSF, IL-1α, IL-1β, MIP-1α, LIF, c-kit ligand, TPO, and flt3 ligand. Cytokines are commercially available from several vendors such as, for example, Genzyme Corp. (Framingham, Mass.), Genentech (South San Francisco, CA), Amgen (Thousand Oaks, CA) and Immunex (Seattle, WA). It is intended, although not always explicitly stated, that molecules having similar biological activity as wild-type or purified cytokines (e.g., recombinantly produced cytokines) are intended to be used within the spirit and scope encompassed by the present invention and therefore are substitutes for wild-type or purified cytokines.

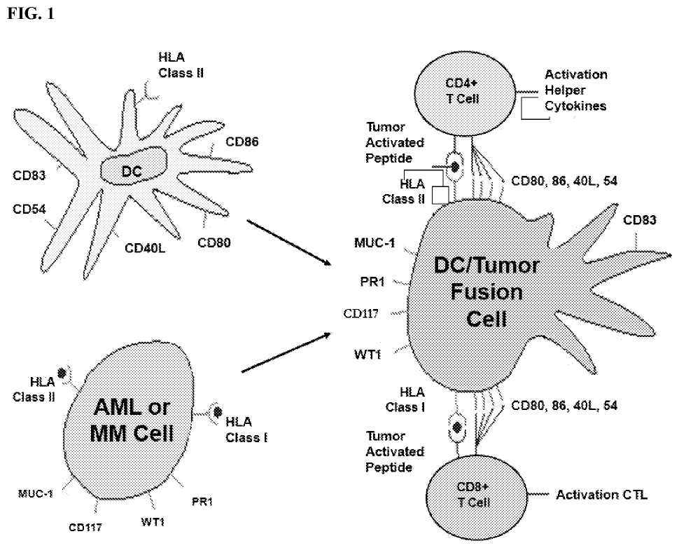

The term “dendritic cell (DC)/tumor fusion vaccine” refers to a hybrid cell or population of cells formed by the fusion of a DC to a non-DC cell or extracellular vesicle, wherein the cell or extracellular vesicle expresses or otherwise comprises a cancer antigen (FIG. 1). A DC expresses immune costimulatory molecules, such as MHC class I and class II molecules, adhesion molecules, and/or a member of the B7 protein family. DCs can be obtained from bone marrow cultures, peripheral blood, spleen or another tissue from a subject. The subject can be the subject that will receive the DC/tumor fusion vaccine. DC progenitors can be obtained and treated with cytokines to produce DCs suitable for use in a DC/tumor fusion vaccine. The non-DC cell can be a cancer cell or a cell that expresses a cancer antigen or extracellular vesicle obtained from the same subject from which the DCs were obtained or from a different subject. In some embodiments, the non-DC cell or extracellular vesicle is modified to express one or more cancer antigens or other immune stimulatory peptides. The DC and non-DC cell can be fused using methods known in the art (see e.g., U.S. Pat. No. 6,652,848; 7,601,342). Additional methods for making and using DC/tumor fusion hybrids are disclosed in U.S. application Ser. No. 12/741,472 (US20100278873); Ser. No. 15/563,127 (US20180078626); Ser. No. 15/563,151 (US20180078650); Ser. No. 15/563,164 (US20180071340); Ser. No. 15/776,369 (US20200323952); Ser. No. 15/563,199 (US20180085350); Ser. No. 15/563,415 (US20180085398); Ser. No. 16/088,977 (US20190125848); U.S. Pat. No. 16,348,567 (US20190269775); U.S. Pat. No. 16,348,564 (US20200000896); and Ser. No. 16/457,578 (US20190326981). In some embodiments, the DC-tumor fusion is a personalized cancer vaccine in which patient derived tumor cells are fused with autologous dendritic cells (DCs), presenting a broad array of antigens that capture the heterogeneity of the cancer cell population, including shared and neoantigens and critical sub-clonal populations.

Variants of a biomarker protein which function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan, 1992, Proc. Natl. Acad. Sci. USA 89:7811-7815; Delgrave et al., 1993, Protein Engineering 6(3):327-331).

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “diagnosing cancer” includes the use of the methods, systems, and code of the present invention to determine the presence or absence of a cancer or subtype thereof in an individual. The term also includes methods, systems, and code for assessing the level of disease activity in an individual.

The term “expression signature” or “signature” refers to a group of one or more coordinately expressed biomarkers related to a measured phenotype. For example, the genes, proteins, metabolites, and the like making up this signature may be expressed in a specific cell lineage, stage of differentiation, or during a particular biological response. The biomarkers can reflect biological aspects of the tumors or cancer cells in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “hypomethylating agent” or “HMA” refers to an agent that reduces or reverses DNA methylation, either at a specific site (e g, a specific CpG island) or generally throughout a genome. In some embodiments, the DNA hypomethylating agent is a DNA methyltransferase inhibitor (DNMTi). The DNMTi may be a small molecule, a biologic, an antisense RNA, a small interfering RNA (siRNA), and combinations thereof. For example, the DNMTi may be a small molecule such as a nucleoside analog. As used herein, a “nucleoside analog” means a molecule that resembles a naturally occurring nucleoside, but which has a chemical or physical modification on the base and/or the sugar moiety, such as a different or additional side group. Such analogs are discussed in, e.g., Scheit, Nucleotide Analogs (John Wiley & Son, 1980) and Uhlman et al. (1990) Chemical Reviews 90:543-584. Non-limiting examples of DNMTi nucleoside analogs include azacitidine (5-azacytidine), decitabine (5-aza-2′-deoxycytidine), FdCyd (5-fluoro-2′-deoxycitidine), DHAC (5,6-dihydro-5-azacytidine), zebularine (1-(β-D-Ribofuranosyl)-1,2-dihydropyrimidin-2-one), fazarabine (1-β-D-arabinofuranosyl-5-azacytosine), 5-aza-2′-deoxycytidine-p-deoxyguanosine, fluorocyclopentenylcytosine, NPEOC-DAC (2-(p-nitrophenyl) ethoxycarbonyl-5-aza-2′-deoxycytidine), CP-4200, MG98, T-dCyd (4′-thio-2′-deoxycytidine), RX-3117 (fluorocyclopentenylcytosine) (Rexahn Pharmaceuticals Inc., Rockville, MD), guadecitabine/SGI-110 (5-aza-2′-deoxycytidine-p-deoxyguanosine) (Astex Pharmaceuticals, Dublin, CA), 5-aza-T-dCyd (5-aza-4′-thio-2′-deoxycytidine), DNA methyltransferase inhibitors (IkerChem, San Sebastian, Spain), EGX-30P (EpiGenX Pharmaceuticals, Santa Barbara, CA), MeTase inhibitor (MethylGene, Montreal, Canada), prodrugs thereof, pharmaceutically acceptable salts thereof, and combinations thereof.

The DNMTi may also be a non-nucleoside analog. Non-limiting examples of DNTMi non-nucleoside analogs include hydralizine, disulfiram, procaine, procainamide, epigallocatechin gallate, psammaplins, RG108 ((S)-2-(1,3-dioxo-1,3-dihydro-isoindol-2-yl)-3-(1H-indol-3-yl)-propionic acid), antineoplaston AS2-1 (Burzynski Research Institute, Houston, TX), mithramycin A, nanaomycin A, SGI-1027, halomon, prodrugs thereof, pharmaceutically acceptable salts thereof, and combinations thereof. Furthermore, the DNMTi may be a biologic. Non-limiting examples of DNTMi biologies include CC-014 (CellCentric, Cambridge, UK), CC-034 (CellCentric), and combinations thereof.

Additionally, the DNMTi may be an antisense RNA. Non-limiting examples of DNTMi antisense RNA include MG-98 (MethylGene, Montreal, Canada). Additional examples of DNMTi are described in PCT/US2012/060663 and EP 3207932, which are incorporated herein by references.

There are several hypomethylating agents for use in treating a clinical indication including azacitidine, decitabine, guadecitabine, 5-Fluro-2′-deoxycytidine, zebularine, CP-4200, RG108, and nanaomycin A. HMAs (e.g. decitabine or guadecitabine) can be combined with other anti-cancer therapies including BH3 mimetics (e.g., venetoclax). This standard of care therapy has shown promising efficacy in high risk AML subjects including elderly, unfit subjects with high risk molecular features (e.g., FLT3, IDH1/2, NPM1, and TP53 mutations) (DiNardo et al. (2019) Blood 133(1):7-17).

Decitabine (2′-deoxy-5-aza-cytidine, CAS No. 2353-33-5) is a cytidine analog having the following structure:

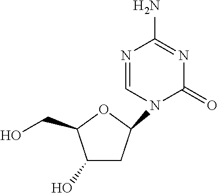 [SMILES: Nc1ncn([C@H]2C[C@H](O)[C@@H](CO)O2)c(=O)n1]

Decitabine is incorporated into the DNA in a cell, but is not a substrate for methylation. Thus, it inhibits DNA methyltransferase activity and is used in the treatment of certain hematological cancers (e.g., myelodysplastic syndromes and AML).

Guadecitabine (2′-deoxy-5-aza-cytidylyl-(3′->5′)-2′-deoxy-guanosine, CAS No. 929901-49-5,) is a dinucleotide antimetabolite of a decitabine having the following structure:

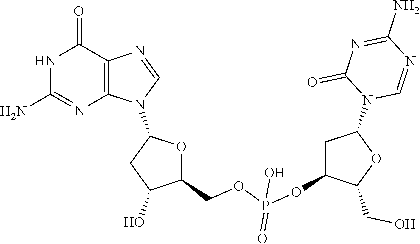 [SMILES: Nc1ncn([C@H]2C[C@H](OP(=O)(O)OC[C@@H]3O[C@@H](n4cnc5c(=O)[nH]c(N)nc54)C[C@H]3O)[C@@H](CO)O2)c(=O)n1]

Guadecitabine, once incorporated into a cell's DNA, inhibits DNA methyltransferase activity, resulting in hypomethylation of the genome.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Dendritic cells (DCs) are professional antigen-presenting cells located in the skin, mucosa and lymphoid tissues. Their main function is to process antigens and present them to T cells to promote immunity to foreign antigens and tolerance to self antigens. They also secrete cytokines to regulate immune responses.

Conventional T cells, also known as Tconv or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+ T lymphocytes. “Naïve Tcons” are CD4+ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, CD39, CD73, 2B4 (CD244), B7.1, B7.2, ILT-2, ILT-4, TIGIT, HHLA2, RGMb, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1, TIM3, and/or RGMb.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP 005009.2 and is shown in Table 1 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well-known and include, for example, mouse PD-1 (NM_008798.2 and NP_032824.1), rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) J. Exp. Med. 192:1027 for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two β sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell. Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

The term “PD-L1” refers to a specific PD-1 ligand. Two forms of human PD-L1 molecules have been identified. One form is a naturally occurring PD-L1 soluble polypeptide, i.e., having a short hydrophilic domain and no transmembrane domain, and is referred to herein as PD-L1S. The second form is a cell-associated polypeptide, i.e., having a transmembrane and cytoplasmic domain, referred to as PD-LIM. The nucleic acid and amino acid sequences of representative human PD-L1 biomarkers regarding PD-LIM are also available to the public at the GenBank database under NM 014143.3 and NP_054862.1. PD-L1 proteins comprise a signal sequence, and an IgV domain and an IgC domain. In addition, nucleic acid and polypeptide sequences of PD-L1 orthologs in organisms other than humans are well-known and include, for example, mouse PD-L1 (NM_021893.3 and NP_068693.1), rat PD-L1 (NM_001191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP 424811.3).

The term “TIM-3” refers to a type I cell-surface glycoprotein that comprises an N-terminal immunoglobulin (Ig)-like domain, a mucin domain with O-linked glycosylations and with N-linked glycosylations close to the membrane, a single transmembrane domain, and a cytoplasmic region with tyrosine phosphorylation motif(s) (see, for example, U.S. Pat. Publ. 2013/0156774). TIM-3 is a member of the T cell/transmembrane, immunoglobulin, and mucin (TIM) gene family. Nucleic acid and polypeptide sequences of human TIM-3 are well-known in the art and are publicly available, for example, as described in NM_032782.4 and NP_116171.3. The term, as described above for useful markers such as PD-L1 and PD-1, encompasses any naturally occurring allelic, splice variants, and processed forms thereof. Typically, TIM-3 refers to human TIM-3 and can include truncated forms or fragments of the TIM-3 polypeptide. In addition, nucleic acid and polypeptide sequences of TIM-3 orthologs in organisms other than humans are well-known and include, for example, mouse TIM-3 (NM_134250.2 and NP_599011.2), chimpanzee TIM-3 (XM_518059.4 and XP_518059.3), dog TIM-3 (NM_001254715.1 and NP_001241644.1), cow TIM-3 (NM_001077105.2 and NP_001070573.1), and rat TIM-3 (NM_001100762.1 and NP_001094232.1). In addition, neutralizing anti-TIM-3 antibodies are well-known in the art (see, at least U.S. Pat. Publ. 2013/0183688, Ngiow et al. (2011) Cancer Res. 71:3540-3551; and antibody 344823 from R&D Biosystems, as well as clones 2C23, 5D12, 2E2, 4A4, and IG5, which are all published and thus publicly available).

TIM-3 was originally identified as a mouse Th1-specific cell surface protein that was expressed after several rounds of in vitro Th1 differentiation, and was later shown to also be expressed on Th17 cells. In humans, TIM-3 is expressed on a subset of activated CD4+ T cells, on differentiated Th1 cells, on some CD8+ T cells, and at lower levels on Th17 cells (Hastings et al. (2009) Eur. J. Immunol. 39:2492-2501). TIM-3 is also expressed on cells of the innate immune system including mouse mast cells, subpopulations of macrophages and dendritic cells (DCs), NK and NKT cells, human monocytes, human dendritic cells, and on murine primary bronchial epithelial cell lines. TIM-3 expression is regulated by the transcription factor T-bet. TIM-3 can generate an inhibitory signal resulting in apoptosis of Th1 and Tc1 cells, and can mediate phagocytosis of apoptotic cells and cross-presentation of antigen. Polymorphisms in TIM-1 and TIM-3 can reciprocally regulate the direction of T-cell responses (Freeman et al. (2010) Immunol. Rev. 235:172-89).

TIM-3 has several known ligands, including galectin-9, phosphatidylserine, and HMGB1. For example, galectin-9 is an S-type lectin with two distinct carbohydrate recognition domains joined by a long flexible linker, and has an enhanced affinity for larger poly-N-acetyllactosamine-containing structures. Galectin-9 does not have a signal sequence and is localized in the cytoplasm. However, it can be secreted and exerts its function by binding to glycoproteins on the target cell surface via their carbohydrate chains (Freeman et al. (2010) Immunol. Rev. 235:172-89). Engagement of TIM-3 by galectin-9 leads to Th1 cell death and a consequent decline in IFN-gamma production. In human AML, self-renewal of AML stem cells is driven by an autocrine TIM3-galectin 9 loop that results in activation of the nuclear factor-κB (NF-κB) and β-catenin pathways (Wolf et al. (2020) Nature Rev. Immunology, 20:173-185). When given in vivo, galectin-9 had beneficial effects in several murine disease models, including an EAE model, a mouse model of arthritis, in cardiac and skin allograft transplant models, and contact hypersensitivity and psoriatic models (Freeman et al. (2010) Immunol. Rev. 235:172-89). Residues important for TIM-3 binding to galectin-9 include TIM-3(44), TIM-3(74), and TIM-3(100), which undergo N- and/or O-glycosylation. It is also known that TIM-3 mediates T-cell dysfunction associated with chronic viral infections (Golden-Mason et al. (2009) J. Virol. 83:9122-9130; Jones et al. (2008) J. Exp. Med. 205:2763-2779) and increases HIV-1-specific T cell responses when blocked ex vivo (Golden-Mason et al. (2009) J. Virol. 83:9122-9130). In addition, in chronic HCV infection, TIM-3 expression was increased on CD4+ and CD8+ T cells, specifically HCV-specific CD8+ cytotoxic T cells (CTLs) in chronic HCV infection and treatment with a blocking monoclonal antibody to TIM-3 reversed HCV-specific T cell exhaustion (Jones et al. (2008) J. Exp. Med. 205:2763-2779).

The term “RGMb” or “DRAGON” refers to a member of the repulsive guidance molecule family, which consists of RGMa, RGMb and RGMc/hemojuvelin (Severyn et al. (2009) Biochem J. 422:393-403). RGMs are glycosylphosphatidylinositol (gpi)-anchored membrane proteins that bind bone morphogenic proteins (BMPs) and neogenin (Conrad et al. (2010) Mol. Cell Neurosci. 43:222-231). BMPs are involved in maintenance of hematopoietic progenitors including support for AML cells (Severyn et al.; Raymond et al. (2014) Oncotarget 5(24):12675-12693 . . . . RGMb also binds to PD-L2, which may mediate immune tolerance (Xiao et al. (2014) J. Exp. Med. 211(5):943-959).

The nucleic acid and amino acid sequences of representative human RGMb biomarkers are well known in the art and are also available to the public at the GenBank database under NM_001012761.2 and NP_001012779.2. RGMb proteins are characterized by common structural elements. In some embodiments, RGMb proteins comprise conserved domains with homology to notch-3, phosphatidylinositol-4-phosphate-5-kinase type II beta, insulin-like growth factor binding protein-2, thrombospondin, ephrin type-B receptor 3 precursor, and Slit-2, all of which are known to influence axonal guidance, neurite outgrowth, and other neuronal developmental functions. The C-terminus of RGMb also contains a hydrophobic domain indicative of a 21 amino acid extracellular GPI anchoring. In addition, nucleic acid and polypeptide sequences of RGMb orthologs in organisms other than humans are well known and include, for example, mouse RGMb (NM_178615.3 and NP_848730.2), chimpanzee RGMb (XM_517848.4 and XP_517848.2), monkey RGMb (NM_001265620.1 and NP_001252549.1), cow RGMb (XM_002689413.2 and XP_002689459.2), chicken RGMb (XM_424860.4 and XP 424860.4), and zebrafish RGMb (NM_001001727.1 and NP_001001727.1).

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-TIM3 antibodies, and/or anti-RGMb antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy), the TIM3-galectin autocrine loop, and/or RGMb-BMP signaling.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein. Indoleamine 2,3,-dioxygenase (IDO) inhibitors, for example INB024360 and 1-MDT can be used as immunotherapeutic agents.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

As used herein, the term “isotype” refers to the antibody class (e.g., IgM, IgG1, IgG2C, IgG2, or IgG4 and the like) that is encoded by heavy chain constant region genes. As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A “kit” is any manufacture (e.g. a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

A “mucin-1 (MUC1) inhibitor” refers to a compound that decreases expression or activity of MUC1. MUC1 is an oncogenic glycoprotein that is aberrantly expressed in many solid tumor and hematological malignancies including MM. MUC1 plays a vital role in supporting key aspects of the malignant phenotype including cell proliferation and self-renewal, resistance to cytotoxic injury and apoptosis, and capacity for migration and tissue invasion. MUC1 is comprised of an N-terminus that is shed into the circulation and a C-terminus that, upon activation, undergoes homodimerization, translocation to the nucleus and interaction with downstream effectors including Wnt/B catenin, NFKB, and the JAK/STAT pathway. MUC1-C can act as an oncoprotein via interactions with receptor tyrosine kinases (RTK) at the cell membrane, thereby promoting activation of these RTKs' pathways (Liu et al. (2014) Blood 123:734-742). In combination with decitabine, the MUC 1 inhibitor GO-203, effectively reduces DNMT1 levels and AML cell survival (Tagde et al. (2016) Oncotarget 2016 Jun. 28; 7(26):38974-38987).

A MUC1 inhibitor decreases expression or activity of MUC1. A decrease in MUC1 activity is defined by a reduction of a biological function of the MUC1. For example, a decrease or reduction in MUC1 expression or biological activity refers to at least a 1%, 2%, 5%, 10%, 15%, 20%, 25%, 30%, 35%, 40%, 45%, 50%, 60%, 70%, 80%, 90% or 100% decrease in MUC1 expression or activity compared to a control.

One MUC1 inhibitor is GO-203 (CAS NO: 1222186-26-6), a D-amino acid peptide (D-arginyl-D-arginyl-D-arginyl-D-arginyl-D-arginyl-D-arginyl-D-arginyl-D-arginyl-D-arginyl-D-cysteinyl-D-glutaminyl-D-cysteinyl-D-arginyl-D-arginyl-D-lysyl-D-asparagine trifluoroacetic acid).

The “normal” level of expression of a biomarker is the level of expression of the biomarker in cells of a subject, e.g., a human subject, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as DC-tumor fusion vaccine, venetoclax (and decitabine in some embodiments), and an immune checkpoint inhibitor combination therapy, and/or evaluate the disease state. A pre-determined biomarker amount and/or activity measurement(s) may be determined in populations of subjects with or without cancer. The pre-determined biomarker amount and/or activity measurement(s) can be a single number, equally applicable to every subject, or the pre-determined biomarker amount and/or activity measurement(s) can vary according to specific subpopulations of subjects. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a subject selection is being assessed. In one embodiment, the pre-determined biomarker amount and/or activity measurement(s) can be obtained from a previous assessment of the same subject. In such a manner, the progress of the selection of the subject can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as esophageal cancer and gastric cancer), development of one or more clinical factors, or recovery from the disease.

The term “response to anti-cancer therapy” or relates to any response of the hyperproliferative disorder (e.g., cancer) to an anti-cancer agent such as a DC-tumor fusion vaccine and a Bcl-2 inhibitor. Hyperproliferative disorder response may be assessed, for example for efficacy, by comparing the size of a tumor or the number of cancerous cells in a subject sample after systemic intervention to the initial size and dimensions. Tumor size and dimensions can be measured by CT, PET, mammogram, ultrasound or palpation. Cell counts can be performed using microscopy or FACS. Responses may also be assessed by caliper measurement or pathological examination of the tumor after biopsy or surgical resection. For hematological cancers such as AML, cell morphology, the abundance of immature cells in the bone marrow (measured, for example, using flow cytometry), and genomic testing to detect mutations and chromosomal abnormalities are assessed. For subjects in remission for a hematologic cancer, genetic analysis and the detection of chromosomal abnormalities are performed to detect cancerous cells, which above a certain threshold indicates minimal residual disease (MRD). Response may be recorded in a quantitative fashion like percentage change in tumor volume or by cell count or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. In some embodiments, clinical efficacy of the therapeutic treatments described herein may be determined by measuring the clinical benefit rate (CBR). The clinical benefit rate is measured by determining the sum of the percentage of subjects who are in complete remission (CR), the number of subjects who are in partial remission (PR) and the number of subjects having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more.

Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor/cancer recurrence. For example, in order to determine appropriate threshold values, a particular cancer therapeutic regimen can be administered to a population of subjects and the outcome can be correlated to biomarker measurements that were determined prior to administration of any cancer therapy. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, e.g. in the sense of reduction of cancerous cells or tumor size or inhibiting tumor growth or cancer cell proliferation. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a cancer or subject will exhibit a favorable response is equivalent to evaluating the likelihood that the cancer or subject will not exhibit favorable response (i.e., will exhibit a lack of response or be non-responsive).

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the anti-immune checkpoint therapy. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42:2159-2164), cell death assays (Weisenthal L M, Shoemaker R H, Marsden J A, Dill P L, Baker J A, Moran E M, Cancer Res 1984; 94:161-173; Weisenthal L M, Lippman M E, Cancer Treat Rep 1985; 69:615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal L M, Contrib Gynecol Obstet 1994; 19:82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic. In some embodiments, the small molecule is a Bcl-2 inhibitor.

The term “specific binding” refers to antibody binding to a predetermined antigen. Typically, the antibody binds with an affinity (K_{D}) of approximately less than 10-7 M, such as approximately less than 10-8 M, 10-9 M or 10-10 M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, acute myeloid leukemia, multiple myeloma, and the like. The term “subject” is interchangeable with “subject.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more anti-cancer agents (e.g., a DC-tumor fusion vaccine and venetoclax) can be greater than the sum of the separate effects of the anticancer agents alone.

The term “T cell” includes CD4+ T cells and CD8+ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

An “underexpression” or “significantly lower level of expression or copy number” of a marker refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with cancer) and preferably, the average expression level or copy number of the marker in several control samples.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the API sequence that can be found within the enhancer (Kang et al. (1992) Science 257:1134).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Tables 1 and 2) are well-known in the art and readily available on publicly available databases, such as the National Center for Biotechnology Information (NCBI). For example, exemplary nucleic acid and amino acid sequences derived from publicly available sequence databases are provided below and include, for example, PCT Publ. WO 2014/022759, which is incorporated herein in its entirety by this reference.

TABLE 1
Human BCL-2 isoform alpha cDNA sequence
SEQ ID NO: 1
>NM_000633.3 Homo sapiens BCL2 apoptosis regulator (BCL2), transcript
variant alpha, mRNA
ACCCCTCGCCGCACCACACACAGCGCGGGCTTCTAGCGCTCGGCACCGGCGGGCCAGGCGCGTCCTGCCT
TCATTTATCCAGCAGCTTTTCGGAAAATGCATTTGCTGTTCGGAGTTTAATCAGAAGAGGATTCCTGCCT
CCGTCCCCGGCTCCTTCATCGTCCCCTCTCCCCTGTCTCTCTCCTGGGGAGGCGTGAAGCGGTCCCGTGG
ATAGAGATTCATGCCTGTGCCCGCGCGTGTGTGCGCGCGTGTAAATTGCCGAGAAGGGGAAAACATCACA
GGACTTCTGCGAATACCGGACTGAAAATTGTAATTCATCTGCCGCCGCCGCTGCCTTTTTTTTTTCTCGA
GCTCTTGAGATCTCCGGTTGGGATTCCTGCGGATTGACATTTCTGTGAAGCAGAAGTCTGGGAATCGATC
TGGAAATCCTCCTAATTTTTACTCCCTCTCCCCGCGACTCCTGATTCATTGGGAAGTTTCAAATCAGCTA
TAACTGGAGAGTGCTGAAGATTGATGGGATCGTTGCCTTATGCATTTGTTTTGGTTTTACAAAAAGGAAA
CTTGACAGAGGATCATGCTGTACTTAAAAAATACAACATCACAGAGGAAGTAGACTGATATTAACAATAC
TTACTAATAATAACGTGCCTCATGAAATAAAGATCCGAAAGGAATTGGAATAAAAATTTCCTGCATCTCA
TGCCAAGGGGGAAACACCAGAATCAAGTGTTCCGCGTGATTGAAGACACCCCCTCGTCCAAGAATGCAAA
GCACATCCAATAAAATAGCTGGATTATAACTCCTCTTCTTTCTCTGGGGGCCGTGGGGTGGGAGCTGGGG
CGAGAGGTGCCGTTGGCCCCCGTTGCTTTTCCTCTGGGAAGGATGGCGCACGCTGGGAGAACAGGGTACG
ATAACCGGGAGATAGTGATGAAGTACATCCATTATAAGCTGTCGCAGAGGGGCTACGAGTGGGATGCGGG
AGATGTGGGCGCCGCGCCCCCGGGGGCCGCCCCCGCACCGGGCATCTTCTCCTCCCAGCCCGGGCACACG
CCCCATCCAGCCGCATCCCGGGACCCGGTCGCCAGGACCTCGCCGCTGCAGACCCCGGCTGCCCCCGGCG
CCGCCGCGGGGCCTGCGCTCAGCCCGGTGCCACCTGTGGTCCACCTGACCCTCCGCCAGGCCGGCGACGA
CTTCTCCCGCCGCTACCGCCGCGACTTCGCCGAGATGTCCAGCCAGCTGCACCTGACGCCCTTCACCGCG
CGGGGACGCTTTGCCACGGTGGTGGAGGAGCTCTTCAGGGACGGGGTGAACTGGGGGAGGATTGTGGCCT
TCTTTGAGTTCGGTGGGGTCATGTGTGTGGAGAGCGTCAACCGGGAGATGTCGCCCCTGGTGGACAACAT
CGCCCTGTGGATGACTGAGTACCTGAACCGGCACCTGCACACCTGGATCCAGGATAACGGAGGCTGGGAT
GCCTTTGTGGAACTGTACGGCCCCAGCATGCGGCCTCTGTTTGATTTCTCCTGGCTGTCTCTGAAGACTC
TGCTCAGTTTGGCCCTGGTGGGAGCTTGCATCACCCTGGGTGCCTATCTGGGCCACAAGTGAAGTCAACA
TGCCTGCCCCAAACAAATATGCAAAAGGTTCACTAAAGCAGTAGAAATAATATGCATTGTCAGTGATGTA
CCATGAAACAAAGCTGCAGGCTGTTTAAGAAAAAATAACACACATATAAACATCACACACACAGACAGAC
ACACACACACACAACAATTAACAGTCTTCAGGCAAAACGTCGAATCAGCTATTTACTGCCAAAGGGAAAT
ATCATTTATTTTTTACATTATTAAGAAAAAAAGATTTATTTATTTAAGACAGTCCCATCAAAACTCCTGT
CTTTGGAAATCCGACCACTAATTGCCAAGCACCGCTTCGTGTGGCTCCACCTGGATGTTCTGTGCCTGTA
AACATAGATTCGCTTTCCATGTTGTTGGCCGGATCACCATCTGAAGAGCAGACGGATGGAAAAAGGACCT
GATCATTGGGGAAGCTGGCTTTCTGGCTGCTGGAGGCTGGGGAGAAGGTGTTCATTCACTTGCATTTCTT
TGCCCTGGGGGCTGTGATATTAACAGAGGGAGGGTTCCTGTGGGGGGAAGTCCATGCCTCCCTGGCCTGA
AGAAGAGACTCTTTGCATATGACTCACATGATGCATACCTGGTGGGAGGAAAAGAGTTGGGAACTTCAGA
TGGACCTAGTACCCACTGAGATTTCCACGCCGAAGGACAGCGATGGGAAAAATGCCCTTAAATCATAGGA
AAGTATTTTTTTAAGCTACCAATTGTGCCGAGAAAAGCATTTTAGCAATTTATACAATATCATCCAGTAC
CTTAAGCCCTGATTGTGTATATTCATATATTTTGGATACGCACCCCCCAACTCCCAATACTGGCTCTGTC
TGAGTAAGAAACAGAATCCTCTGGAACTTGAGGAAGTGAACATTTCGGTGACTTCCGCATCAGGAAGGCT
AGAGTTACCCAGAGCATCAGGCCGCCACAAGTGCCTGCTTTTAGGAGACCGAAGTCCGCAGAACCTGCCT
GTGTCCCAGCTTGGAGGCCTGGTCCTGGAACTGAGCCGGGGCCCTCACTGGCCTCCTCCAGGGATGATCA
ACAGGGCAGTGTGGTCTCCGAATGTCTGGAAGCTGATGGAGCTCAGAATTCCACTGTCAAGAAAGAGCAG
TAGAGGGGTGTGGCTGGGCCTGTCACCCTGGGGCCCTCCAGGTAGGCCCGTTTTCACGTGGAGCATGGGA
GCCACGACCCTTCTTAAGACATGTATCACTGTAGAGGGAAGGAACAGAGGCCCTGGGCCCTTCCTATCAG
AAGGACATGGTGAAGGCTGGGAACGTGAGGAGAGGCAATGGCCACGGCCCATTTTGGCTGTAGCACATGG
CACGTTGGCTGTGTGGCCTTGGCCCACCTGTGAGTTTAAAGCAAGGCTTTAAATGACTTTGGAGAGGGTC
ACAAATCCTAAAAGAAGCATTGAAGTGAGGTGTCATGGATTAATTGACCCCTGTCTATGGAATTACATGT
AAAACATTATCTTGTCACTGTAGTTTGGTTTTATTTGAAAACCTGACAAAAAAAAAGTTCCAGGTGTGGA
ATATGGGGGTTATCTGTACATCCTGGGGCATTAAAAAAAAAATCAATGGTGGGGAACTATAAAGAAGTAA
CAAAAGAAGTGACATCTTCAGCAAATAAACTAGGAAATTTTTTTTTCTTCCAGTTTAGAATCAGCCTTGA
AACATTGATGGAATAACTCTGTGGCATTATTGCATTATATACCATTTATCTGTATTAACTTTGGAATGTA
CTCTGTTCAATGTTTAATGCTGTGGTTGATATTTCGAAAGCTGCTTTAAAAAAATACATGCATCTCAGCG
TTTTTTTGTTTTTAATTGTATTTAGTTATGGCCTATACACTATTTGTGAGCAAAGGTGATCGTTTTCTGT
TTGAGATTTTTATCTCTTGATTCTTCAAAAGCATTCTGAGAAGGTGAGATAAGCCCTGAGTCTCAGCTAC
CTAAGAAAAACCTGGATGTCACTGGCCACTGAGGAGCTTTGTTTCAACCAAGTCATGTGCATTTCCACGT
CAACAGAATTGTTTATTGTGACAGTTATATCTGTTGTCCCTTTGACCTTGTTTCTTGAAGGTTTCCTCGT
CCCTGGGCAATTCCGCATTTAATTCATGGTATTCAGGATTACATGCATGTTTGGTTAAACCCATGAGATT
CATTCAGTTAAAAATCCAGATGGCAAATGACCAGCAGATTCAAATCTATGGTGGTTTGACCTTTAGAGAG
TTGCTTTACGTGGCCTGTTTCAACACAGACCCACCCAGAGCCCTCCTGCCCTCCTTCCGCGGGGGCTTTC
TCATGGCTGTCCTTCAGGGTCTTCCTGAAATGCAGTGGTGCTTACGCTCCACCAAGAAAGCAGGAAACCT
GTGGTATGAAGCCAGACCTCCCCGGCGGGCCTCAGGGAACAGAATGATCAGACCTTTGAATGATTCTAAT
TTTTAAGCAAAATATTATTTTATGAAAGGTTTACATTGTCAAAGTGATGAATATGGAATATCCAATCCTG
TGCTGCTATCCTGCCAAAATCATTTTAATGGAGTCAGTTTGCAGTATGCTCCACGTGGTAAGATCCTCCA
AGCTGCTTTAGAAGTAACAATGAAGAACGTGGACGTTTTTAATATAAAGCCTGTTTTGTCTTTTGTTGTT
GTTCAAACGGGATTCACAGAGTATTTGAAAAATGTATATATATTAAGAGGTCACGGGGGCTAATTGCTGG
CTGGCTGCCTTTTGCTGTGGGGTTTTGTTACCTGGTTTTAATAACAGTAAATGTGCCCAGCCTCTTGGCC
CCAGAACTGTACAGTATTGTGGCTGCACTTGCTCTAAGAGTAGTTGATGTTGCATTTTCCTTATTGTTAA
AAACATGTTAGAAGCAATGAATGTATATAAAAGCCTCAACTAGTCATTTTTTTCTCCTCTTCTTTTTTTT
CATTATATCTAATTATTTTGCAGTTGGGCAACAGAGAACCATCCCTATTTTGTATTGAAGAGGGATTCAC
ATCTGCATCTTAACTGCTCTTTATGAATGAAAAAACAGTCCTCTGTATGTACTCCTCTTTACACTGGCCA
GGGTCAGAGTTAAATAGAGTATATGCACTTTCCAAATTGGGGACAAGGGCTCTAAAAAAAGCCCCAAAAG
GAGAAGAACATCTGAGAACCTCCTCGGCCCTCCCAGTCCCTCGCTGCACAAATACTCCGCAAGAGAGGCC
AGAATGACAGCTGACAGGGTCTATGGCCATCGGGTCGTCTCCGAAGATTTGGCAGGGGCAGAAAACTCTG
GCAGGCTTAAGATTTGGAATAAAGTCACAGAATTAAGGAAGCACCTCAATTTAGTTCAAACAAGACGCCA
ACATTCTCTCCACAGCTCACTTACCTCTCTGTGTTCAGATGTGGCCTTCCATTTATATGTGATCTTTGTT
TTATTAGTAAATGCTTATCATCTAAAGATGTAGCTCTGGCCCAGTGGGAAAAATTAGGAAGTGATTATAA
ATCGAGAGGAGTTATAATAATCAAGATTAAATGTAAATAATCAGGGCAATCCCAACACATGTCTAGCTTT
CACCTCCAGGATCTATTGAGTGAACAGAATTGCAAATAGTCTCTATTTGTAATTGAACTTATCCTAAAAC
AAATAGTTTATAAATGTGAACTTAAACTCTAATTAATTCCAACTGTACTTTTAAGGCAGTGGCTGTTTTT
AGACTTTCTTATCACTTATAGTTAGTAATGTACACCTACTCTATCAGAGAAAAACAGGAAAGGCTCGAAA
TACAAGCCATTCTAAGGAAATTAGGGAGTCAGTTGAAATTCTATTCTGATCTTATTCTGTGGTGTCTTTT
GCAGCCCAGACAAATGTGGTTACACACTTTTTAAGAAATACAATTCTACATTGTCAAGCTTATGAAGGTT
CCAATCAGATCTTTATTGTTATTCAATTTGGATCTTTCAGGGATTTTTTTTTTAAATTATTATGGGACAA
AGGACATTTGTTGGAGGGGTGGGAGGGAGGAAGAATTTTTAAATGTAAAACATTCCCAAGTTTGGATCAG
GGAGTTGGAAGTTTTCAGAATAACCAGAACTAAGGGTATGAAGGACCTGTATTGGGGTCGATGTGATGCC
TCTGCGAAGAACCTTGTGTGACAAATGAGAAACATTTTGAAGTTTGTGGTACGACCTTTAGATTCCAGAG
ACATCAGCATGGCTCAAAGTGCAGCTCCGTTTGGCAGTGCAATGGTATAAATTTCAAGCTGGATATGTCT
AATGGGTATTTAAACAATAAATGTGCAGTTTTAACTAACAGGATATTTAATGACAACCTTCTGGTTGGTA
GGGACATCTGTTTCTAAATGTTTATTATGTACAATACAGAAAAAAATTTTATAAAATTAAGCAATGTGAA
ACTGAATTGGAGAGTGATAATACAAGTCCTTTAGTCTTACCCAGTGAATCATTCTGTTCCATGTCTTTGG
ACAACCATGACCTTGGACAATCATGAAATATGCATCTCACTGGATGCAAAGAAAATCAGATGGAGCATGA
ATGGTACTGTACCGGTTCATCTGGACTGCCCCAGAAAAATAACTTCAAGCAAACATCCTATCAACAACAA
GGTTGTTCTGCATACCAAGCTGAGCACAGAAGATGGGAACACTGGTGGAGGATGGAAAGGCTCGCTCAAT
CAAGAAAATTCTGAGACTATTAATAAATAAGACTGTAGTGTAGATACTGAGTAAATCCATGCACCTAAAC
CTTTTGGAAAATCTGCCGTGGGCCCTCCAGATAGCTCATTTCATTAAGTTTTTCCCTCCAAGGTAGAATT
TGCAAGAGTGACAGTGGATTGCATTTCTTTTGGGGAAGCTTTCTTTTGGTGGTTTTGTTTATTATACCTT
CTTAAGTTTTCAACCAAGGTTTGCTTTTGTTTTGAGTTACTGGGGTTATTTTTGTTTTAAATAAAAATAA
GTGTACAATAAGTGTTTTTGTATTGAAAGCTTTTGTTATCAAGATTTTCATACTTTTACCTTCCATGGCT
CTTTTTAAGATTGATACTTTTAAGAGGTGGCTGATATTCTGCAACACTGTACACATAAAAAATACGGTAA
GGATACTTTACATGGTTAAGGTAAAGTAAGTCTCCAGTTGGCCACCATTAGCTATAATGGCACTTTGTTT
GTGTTGTTGGAAAAAGTCACATTGCCATTAAACTTTCCTTGTCTGTCTAGTTAATATTGTGAAGAAAAAT
AAAGTACAGTGTGAGATACTG
Human BCL-2 isoform alpha amino acid sequence
SEQ ID NO: 2
>NP_000624.2 apoptosis regulator Bcl-2 isoform alpha [Homo sapiens]
MAHAGRTGYDNREIVMKYIHYKLSQRGYEWDAGDVGAAPPGAAPAPGIFSSQPGHTPHPAASRDPVARTS
PLQTPAAPGAAAGPALSPVPPVVHLTLRQAGDDFSRRYRRDFAEMSSQLHLTPFTARGRFATVVEELFRD
GVNWGRIVAFFEFGGVMCVESVNREMSPLVDNIALWMTEYLNRHLHTWIQDNGGWDAFVELYGPSMRPLF
DFSWLSLKTLLSLALVGACITLGAYLGHK
Human BCL-2 isoform beta cDNA sequence
SEQ ID NO: 3
>NM_000657.3 Homo sapiens BCL2 apoptosis regulator (BCL2), transcript
variant beta, mRNA
ACCCCTCGCCGCACCACACACAGCGCGGGCTTCTAGCGCTCGGCACCGGCGGGCCAGGCGCGTCCTGCCT
TCATTTATCCAGCAGCTTTTCGGAAAATGCATTTGCTGTTCGGAGTTTAATCAGAAGAGGATTCCTGCCT
CCGTCCCCGGCTCCTTCATCGTCCCCTCTCCCCTGTCTCTCTCCTGGGGAGGCGTGAAGCGGTCCCGTGG
ATAGAGATTCATGCCTGTGCCCGCGCGTGTGTGCGCGCGTGTAAATTGCCGAGAAGGGGAAAACATCACA
GGACTTCTGCGAATACCGGACTGAAAATTGTAATTCATCTGCCGCCGCCGCTGCCTTTTTTTTTTCTCGA
GCTCTTGAGATCTCCGGTTGGGATTCCTGCGGATTGACATTTCTGTGAAGCAGAAGTCTGGGAATCGATC
TGGAAATCCTCCTAATTTTTACTCCCTCTCCCCGCGACTCCTGATTCATTGGGAAGTTTCAAATCAGCTA
TAACTGGAGAGTGCTGAAGATTGATGGGATCGTTGCCTTATGCATTTGTTTTGGTTTTACAAAAAGGAAA
CTTGACAGAGGATCATGCTGTACTTAAAAAATACAACATCACAGAGGAAGTAGACTGATATTAACAATAC
TTACTAATAATAACGTGCCTCATGAAATAAAGATCCGAAAGGAATTGGAATAAAAATTTCCTGCATCTCA
TGCCAAGGGGGAAACACCAGAATCAAGTGTTCCGCGTGATTGAAGACACCCCCTCGTCCAAGAATGCAAA
GCACATCCAATAAAATAGCTGGATTATAACTCCTCTTCTTTCTCTGGGGGCCGTGGGGTGGGAGCTGGGG
CGAGAGGTGCCGTTGGCCCCCGTTGCTTTTCCTCTGGGAAGGATGGCGCACGCTGGGAGAACAGGGTACG
ATAACCGGGAGATAGTGATGAAGTACATCCATTATAAGCTGTCGCAGAGGGGCTACGAGTGGGATGCGGG
AGATGTGGGCGCCGCGCCCCCGGGGGCCGCCCCCGCACCGGGCATCTTCTCCTCCCAGCCCGGGCACACG
CCCCATCCAGCCGCATCCCGGGACCCGGTCGCCAGGACCTCGCCGCTGCAGACCCCGGCTGCCCCCGGCG
CCGCCGCGGGGCCTGCGCTCAGCCCGGTGCCACCTGTGGTCCACCTGACCCTCCGCCAGGCCGGCGACGA
CTTCTCCCGCCGCTACCGCCGCGACTTCGCCGAGATGTCCAGCCAGCTGCACCTGACGCCCTTCACCGCG
CGGGGACGCTTTGCCACGGTGGTGGAGGAGCTCTTCAGGGACGGGGTGAACTGGGGGAGGATTGTGGCCT
TCTTTGAGTTCGGTGGGGTCATGTGTGTGGAGAGCGTCAACCGGGAGATGTCGCCCCTGGTGGACAACAT
CGCCCTGTGGATGACTGAGTACCTGAACCGGCACCTGCACACCTGGATCCAGGATAACGGAGGCTGGGTA
GGTGCACTTGGTGATGTGAGTCTGGGCTGAGGCCACAGGTCCGAGATGCGGGGGTTGGAGTGCGGGTGGG
CTCCTGGGGCAATGGGAGGCTGTGGAGCCGGCGAAATAAAATCAGAGTTGTTGCT
Human BCL-2 isoform beta amino acid sequence
SEQ ID NO: 4
>NP_000648.2 apoptosis regulator Bcl-2 isoform beta [Homo sapiens]
MAHAGRTGYDNREIVMKYIHYKLSQRGYEWDAGDVGAAPPGAAPAPGIFSSQPGHTPHPAASRDPVARTS
PLQTPAAPGAAAGPALSPVPPVVHLTLRQAGDDESRRYRRDFAEMSSQLHLTPFTARGRFATVVEELFRD
GVNWGRIVAFFEFGGVMCVESVNREMSPLVDNIALWMTEYLNRHLHTWIQDNGGWVGALGDVSLG

### II. Subjects

In one embodiment, the subject to be administered a DC-tumor fusion vaccine and a Bcl-2 inhibitor is a mammal (e.g., mouse, rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer. In some embodiments, the subject is administered a DC-tumor fusion vaccine, a Bcl-2 inhibitor, and a hypomethylating agent (HMA).

In another embodiment of the methods of the present invention, the subject has not undergone treatment, such as chemotherapy, radiation therapy, targeted therapy, and/or anti-immune checkpoint therapy. In still another embodiment, the subject has undergone treatment, such as chemotherapy, radiation therapy, targeted therapy, and/or anti-immune checkpoint therapy.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the subject. In other embodiments, the subject is in remission.

The methods of the present invention can be used to treat many different cancers in subjects such as those described herein.

### III. Anti-Cancer Therapies

a. Dendritic Cell-Tumor Cell Fusion Vaccine

The present invention provides a dendritic cell (DC)/tumor fusion vaccine comprising a dendritic cell fused to a cancer cell. The cancer cells may be derived from a solid or hematological cancer (e.g., acute myeloid leukemia (AML)). In one embodiment, the cancer cells are derived from a subject. In another embodiment, the cancer cells are derived from a cancer cell line. The cancer cells may be derived from the subject who is treated with the DC/tumor fusion vaccine. The cancer cells may also be derived from a different subject who is not treated with the DC/tumor fusion vaccine. The cancer cells may be derived from a cancer that is the same type as the cancer treated with the DC/tumor fusion vaccine. The cancer cells may also be derived from a cancer that is a different type from the cancer treated with the DC/tumor fusion vaccine.

### Dendritic Cells

DCs can be obtained from bone marrow cultures, peripheral blood, spleen, or any other appropriate tissue of a mammal using protocols known in the art. Bone marrow contains DC progenitors, which, upon treatment with cytokines, such as granulocyte-macrophage colony-stimulating factor (“GM-CSF”) and interleukin 4 (“IL-4”), proliferate and differentiate into DCs. Tumor necrosis cell factor (TNF) is optionally used alone or in conjunction with GM-CSF and/or IL-4 to promote maturation of DCs. DCs obtained from bone marrow are relatively immature (as compared to, for instance, spleen DCs). GMCSF/IL-4 stimulated DC express MHC class I and class II molecules, B7-1, B7-2, ICAM, CD40 and variable levels of CD83. These immature DCs are more amenable to fusion (or antigen uptake) than the more mature DCs found in spleen, whereas more mature DCs are relatively more effective antigen presenting cells. Peripheral blood also contains relatively immature DCs or DC progenitors, which can propagate and differentiate in the presence of appropriate cytokines such as GM-CSF and—which can also be used in fusions. Preferably, the DCs are obtained from peripheral blood. For example, the DCs are obtained from the subjects' peripheral blood after it has been documented that the subject is in complete remission.

The DC can be made hyperactive prior to fusion or after fusion. DCs can be made hyperactive by any method know in the art. For example, DCs are made hyperactive by contacting the DC or DC fusion with a priming agent followed by an activating agent. Exemplary priming agents include CpG DNA or LPS. Activating agents include for example oxidized phospholipids.

In some embodiments, DCs have sufficient viability prior to fusion, such as at least 70%, at least 75%, at least 80%, or greater.

Prior to fusion, the population of the DCs are generally free of components used during the production, e.g., cell culture components and substantially free of mycoplasm, endotoxin, and microbial contamination. Preferably, the population of DCs has less than 10, 5, 3, 2, or 1 CFU/swab. Most preferably, the population of DCs has 0 CFU/swab.

Prior to fusion, the population of vehicles expressing tumor antigens is generally free of components used during the isolation and substantially free of mycoplasm endotoxin, and microbial contamination. Preferably, the cell population has less than 10, 5, 3, 2, or 1 CFU/swab. Most preferably the population of cells has 0 CFU/swab. The endotoxin level in the population of cells is less than 20 EU/mL, less than 10 EU/mL or less than 5 EU/mL.

Prior to fusion, the population of tumor organoids or spheroids are generally free of components used during the isolation and substantially free of mycoplasm, endotoxin, and microbial contamination. Preferably, the cell population has less than 10, 5, 3, 2, or 1 CFU/swab. Most preferably, the population of cells has 0 CFU/swab. The endotoxin level in the population of cells is generally less than 20 EU/mL, less than 10 EU/mL, or less than 5 EU/mL.

### Cancer Cells

The cancer, cells contemplated for use in connection with the present invention include, but are not limited to, cancer cells from breast cancer cells, ovarian cancer cells, pancreatic cancer cells, prostate gland cancer cells, renal cancer cells, lung cancer cells, urothelial cancer cells, colon cancer cells, rectal cancer cells, or hematological cancer cells. For example, hematological cancer cells include, but are not limited to, acute myeloid leukemia cells, acute lymphoid leukemia cells, multiple myeloma cells, and non-Hodgkin's lymphoma cells. Moreover, those skilled in the art would recognize that any tumor cell may be used in any of the methods of the present invention. Throughout the specification the terms “cancer” and “tumor” are used interchangeably unless indicated otherwise.

In some aspects, the tumor cells used in producing the cDNA expression library or fusions in accordance with the methods of the present invention include tumor cells obtained directly from a subject. Alternatively, tumor cells obtained from a subject may be cultured in vitro, prior to producing the cDNA library or fusion. Culturing the tumor cells is particularly useful when a sufficient number of tumor cells cannot be obtained from the subject sample. Any in vitro culturing technique may be utilized. Preferably, three dimensional (3D) culturing techniques are utilized to produce spheroids or organoid tumor cultures. Cells grown in 3D cultures systems to produce spheroids or organoids more closely resemble in vivo tissue in terms of cellular communication the development of extracellular matrices and tumor associated antigens.

3D culturing methods to produce tumor spheroids or organoids are well-known in the art. For example, the 3D culturing methods may utilize scaffold techniques or scaffold-free techniques.

Scaffold techniques include the use of solid scaffolds, hydrogels and other materials. Hydrogels are composed of interconnected pores with high water retention, which enables efficient transport of, e.g., nutrients and gases. Several different types of hydrogels from natural and synthetic materials are available for 3D cell culture, including, e.g., animal ECM extract hydrogels, protein hydrogels, peptide hydrogels, polymer hydrogels, and wood-based nanocellulose hydrogels.

Scaffold-free techniques employ another approach independent from the use of a scaffold. Scaffold-free methods include, for example, the use of low adhesion plates, hanging drop plates, micropatterned surfaces, rotating bioreactors, magnetic levitation, and/or magnetic 3D bioprinting.

In one embodiment, the subject has undergone therapy for the cancer. In another embodiment, the subject is in post-chemotherapy induced remission. In still another embodiment, the subject has had surgery to remove all or part of a tumor. For example, a multiple myeloma subject can have an autologous stem cell transplant 30 to 100 days prior to the administration of the DC/tumor fusion vaccine. If the subject has renal cell carcinoma, the subject may have a de-bulking nephrectomy prior to the administration of the DC/tumor fusion vaccine. If the subject has AML, then administration of the DC/tumor fusion vaccine follows induction of a complete remission with chemotherapy.

### Cancer Cell Isolation and Purification

In some embodiments, the cancer cells are derived from a subject. Isolation and purification of cancer cell from various cancer tissues such as surgical cancer tissues, ascites or carcinous hydrothorax is a common process to obtain the purified cancer cells. Cancer cells may be purified from fresh biopsy samples from cancer subjects or animal cancer models. The biopsy samples often contain a heterogeneous population of cells that include normal tissue or cells, blood, and cancer cells. Preferably, a purified cancer cell composition can have greater than 70%, 75%, 80%, 85%, 90%, 95%, 99%, or more, or any range in between or any value in between, total viable cancer cells. To purify cancer cells from the heterogeneous population, a number of methods can be used.

In one embodiment, laser microdissection is used to isolate cancer cells. Cancer cells of interest can be carefully dissected from thin tissue slices prepared for microscopy. In this method, the tissue section is coated with a thin plastic film and an area containing the selected cells is irradiated with a focused infrared laser beam pulse. This melts a small circle in the plastic film, causing cell binding underneath. Those captured cells are removed for additional analysis. This technique is good for separating and analyzing cells from different parts of a tumor, which allows for a comparison of their similar and distinct properties. It was used recently to analyze pituitary cells from dissociated tissues and from cultured populations of heterogeneous pituitary, thyroid, and carcinoid tumor cells, as well as analyzing single cells found in various sarcomas.

In another embodiment, fluorescence activated cell sorting (FACS), also referred to as flow cytometry, is used to sort and analyze the different cell populations. Cells having a cellular marker or other specific marker of interest are tagged with an antibody, or typically a mixture of antibodies, that bind the cellular markers. Each antibody directed to a different marker is conjugated to a detectable molecule, particularly a fluorescent dye that may be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and related lineage cells is well-known in the art and described in, for example, U.S. Pat. Nos. 5,137,809; 5,750,397; 5,840,580; 6,465,249; Manz et al. (202) Proc. Natl. Acad. Sci. U.S.A. 99:11872-11877; and Akashi et al. (200) Nature 404:193-197. General guidance on fluorescence activated cell sorting is described in, for example, Shapiro (2003) Practical Flow Cytometry, 4th Ed., Wiley-Liss (2003) and Ormerod (2000) Flow Cytometry: A Practical Approach, 3rd Ed., Oxford University Press.

Another method of isolating useful cell populations involves a solid or insoluble substrate to which is bound antibodies or ligands that interact with specific cell surface markers. In immunoadsorption techniques, cells are contacted with the substrate (e.g., column of beads, flasks, magnetic particles, etc.) containing the antibodies and any unbound cells removed. Immunoadsorption techniques may be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads may be readily isolated by a magnetic separator (see, e.g., Kato and Radbruch (1993) Cytometry 14:384-92). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations. One of the most common uses of this technology is for isolating circulating tumor cells (CTCs) from the blood of breast, NSC lung cancer, prostate and colon cancer subjects using an antibody against EpCAM, a cell surface glycoprotein that has been found to be highly expressed in epithelial cancers.

FACS and most batch wise immunoadsorption techniques may be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that may be employed is use of antibody/complement treatment or immunotoxins to remove unwanted cells.

In still another embodiment, microfluidics is used to isolate cancer cells. This method used a microfluidic chip with a spiral channel that can isolate circulating tumor cells (CTCs) from blood based upon their size. A sample of blood is pumped into the device and as cells flow through the channel at high speeds, the inertial and centrifugal forces cause smaller cells to flow along the outer wall while larger cells, including CTCs, flow along the inner wall. Researchers have used this chip technology to isolate CTCs from the blood of subjects with metastatic lung or breast cancer.

Fluorescent nanodiamonds (FNDs), according to a recently published article (Lin et al. Small (2015) 11:4394-4402), can be used to label and isolate slow-proliferating/quiescent cancer stem cells, which, according to study authors, have been difficult to isolate and track over extended time periods using traditional fluorescent markers. It was concluded that nanoparticles do not cause DNA damage or impair cell growth, and that they outperformed EdU and CFSE fluorescent labels in terms of long-term tracking capability.

It is to be understood that the purification or isolation of cells also includes combinations of the methods described above. A typical combination may comprise an initial procedure that is effective in removing the bulk of unwanted cells and cellular material. A second step may include isolation of cells expressing a marker common to one or more of the progenitor cell populations by immunoadsorption on antibodies bound to a substrate. An additional step providing higher resolution of different cell types, such as FACS sorting with antibodies to a set of specific cellular markers, may be used to obtain substantially pure populations of the desired cells.

### DC/Tumor Fused Cells

The dendritic cells can be fused with tumor cells or extracellular vesicles (see U.S. Pat. No. 6,652,848; U.S. application Ser. No. 16/088,977 (US20190125848). The fusion product can be used directly after the fusion process (e.g., in antigen discovery screening methods or in therapeutic methods) or after a short culture period.

The DC/tumor fusion cells can be irradiated prior to clinical use. Irradiation induces expression of cytokines, which promotes immune effector cell activity. Irradiation also prevents the cells from replicating, thereby reducing or eliminating any risk of oncogenesis.

In the event that the fused DCs lose certain DC characteristics, such as expression of the APC-specific T-cell stimulating molecules, primary fused cells can be re-fused with dendritic cells to restore the DC phenotype. The re-fused cells (i.e., secondary fused cells) are found to be highly potent APCs. The fused cells can be re-fused with the dendritic or non-dendritic parental cells as many times as desired.

Cell fusions that express MHC class II molecules, B7, adhesion molecules characteristic of antigen presenting cells, or other desired T-cell stimulating molecules can also be selected by panning or fluorescence-activated cell sorting with antibodies against these molecules.

Fusion can be carried out with well-known methods, such as those using polyethylene glycol (“PEG”) or electrofusion. Alternatively, the cDNA-NC/NPs or cDNA vesicles or tumor organoids or spheroids can fuse with DCs in the absence of PEG or electrofusion. DCs are autologous or allogeneic (see. e.g., U.S. Pat. No. 6,653,848, which is herein incorporated by reference in its entirety).

The ratio of DCs to MHC I/II null cells expressing tumor antigens in fusion can vary from 1:100 to 1000:1, with a ratio higher than 1:1 being preferred. Preferably, the ratio is 1:1, 5:1, or 10:1. Most preferably, the ratio of DCs to cells is 10:1 or 3:1.

Alternatively, the ratio of DCs to cDNA-NC/NPs encoding, expressing, or presenting tumor antigens can vary from about 2×10⁵ to about 1×10⁶ DCs to about 0.2 to 1.24 μg cDNA-NC/NPs. In some such embodiments the amount of DCs is 2×10⁵ and the amount of cDNA-NC/NPs is 0.2 μg (Kranz et al. (2016) Nature, 534:396). In yet a further alternative, the ratio of DCs to cDNA-vesicles encoding, expressing, or presenting tumor antigens can vary from about 2×10⁵ to about 1×10⁶ DCs to about 0.2 to 20 μg cDNA-vesicles. In some such embodiments the amount of DCs is 2×10⁵ and the amount of cDNA-vesicles is 20 μg (Ding G et al. (2015) Oncotarget 6:29877).

After fusion, unfused DCs usually die off in a few days in culture, and the fused cells can be separated from the unfused, parental, non-dendritic cells by the following two methods, both of which yield fused cells of approximately 50% or higher purity, i.e., the fused cell preparations contain less than 50%, and often less than 30%, unfused cells.

### Isolation of Fused Cells

Specifically, one method of separating unfused cells/vehicles from fused cells is based on the different adherence properties between the fused cells and the vehicles or MHC I/II null cells expressing tumor antigens. It has been found that the fused cells are generally lightly adherent to tissue culture containers. Thus, if the cells expressing tumor antigens are much more adherent, the post-fusion cell mixtures can be cultured in an appropriate medium for a short period of time (e.g., 5-1 0 days). Subsequently, cell fusions can be gently dislodged and aspirated off while the MHC I/II null cells expressing tumor antigens or are firmly attached to the tissue culture containers. Conversely, if the cells expressing tumor antigens are in suspension, after the culture period, they can be gently aspirated off while leaving the DC fusions loosely attached to the containers. Alternatively, the hybrids are used directly without an in vitro cell culturing step.

The cell fusions obtained by the above-described methods typically retain the phenotypic characteristics of DCs. For instance, these fusions express T-cell stimulating molecules such as MHC class II protein, B7-L B7-2, and adhesion molecules characteristic of APCs such as ICAM-I. The fusions also continue to express cell-surface antigens of the tumor cells such as MUC1, and are therefore useful for inducing immunity against the cell surface antigens.

In the event that the fusions lose certain DC characteristics such as expression of the APC-specific T-cell stimulating molecules, they (i.e., primary fusions) can be re-fused with dendritic cells to restore the DC phenotype. The re-fused cells (i.e., secondary fusions) are found to be highly potent APCs, and in some cases, have even less tumorigenicity than primary fusions. The fusions can be re-fused with the dendritic cell as many times as desired. The DCs can be made hyperactive prior to or after re-fusion.

The cell fusions can be frozen before administration. The fusions can be frozen in a solution containing 10% DMSO in 90% heat inactivated autologous plasma.

b. Blc-2 Inhibitors

The present invention is based on the surprising discovery that a DC/tumor fusion vaccine rescues the immune response against a cancer that is muted by anti-Bcl-2 therapy. Bcl-2 inhibitors prevent or reduce the ability of the Bcl-2 polypeptide to bind to BAX and BAK, thereby promoting apoptosis. Accordingly, in aspects encompassed by the present invention, a DC/tumor fusion vaccine and a Bcl-2 inhibitor are administered to subject in need thereof. Bcl-2 inhibitors can be antisense nucleic acids, small molecules, or peptides. As defined above, Bcl-2 inhibitors can be any small molecule, protein (including but not limited to antibodies), nucleic acid, or any other molecule or composition that binds to a Bcl-2 family member (e.g., Bcl-2, Bcl-XL, and Bcl-w, and antagonizes the activity of the Bcl-2 family member protein or nucleic acid encoding the protein, for example, inhibiting Bcl-2 from binding or otherwise activating BAX and BAK.

In some embodiments, the Bcl-2 inhibitor is a BH3 mimetic. Venetoclax, for example is a BH3 mimetic currently used for treating cancer (e.g., AML). In some embodiments, the Bcl-2 inhibitor is an antisense oligonucleotide. Other Bcl-2 inhibitors are contemplated as well, including those undergoing clinical trials or in preclinical development, such as BGB-11417, G3139, and oblimersen.

Bcl-2 inhibitors can be formulated to increase bioavailability (e.g., oral bioavailability). For example, supersaturated lipid-based formulations can improve oral availability of some drugs, including ventoclax (Kochl et al. (2020) Pharmaceutics 12(6):564), and oil-based formulations of BH3 mimetics have shown promise in cancer treatment (Mullard (2016). Nat. Rev. Drug Discov. 15, 147. In some embodiments, the BH3 can be formulated as a salt (e.g., obatoclax mesylate).

c. Additional Anti-Cancer Therapeutics

In some embodiments, a subject is administered a DC/tumor fusion vaccine, a Bcl-2 inhibitor, and an additional anti-cancer therapeutic. In some embodiments, the additional anti-cancer therapeutic is a hypomethylating agent. Examples of hypomethylating agents include, but are not limited to, decitabine, guadecitabine, 5-azacytidine, and 5-fluro-2′-deoxycytidine.

In some embodiments, a DC/tumor fusion vaccine and a Bcl-2 inhibitor can be used in conjunction with a MUC-1 inhibitor, such as GO-203 to treat a cancer.

An inhibitor of indoleamine 2,3-dioxygenase (IDO) can be used in conjuction with a DC/tumor fusion vaccine and a Bcl-2 inhibitor to treat cancer.

In some embodiments, a checkpoint inhibitor can be used in conjunction with a DC/tumor fusion vaccine and a Bcl-2 inhibitor to treat cancer. Examples of checkpoint inhibitors include, but are not limited to, PD-1, PD-L1, TIM-3, and RGMb inhibitors.

In some embodiments, an immunomodulatory agent can be used in conjunction with a DC/tumor fusion vaccine and a Bcl-2 inhibitor to treat cancer. Examples of immunomodulatory agents include, but are not limited to, lenalidomide, pomalinomide, and apremilast.

Cytokines can also be used in conjunction with a DC/tumor fusion vaccine and a Bcl-2 inhibitor to treat cancer. For example, GM-CSF can be used with a a DC/tumor fusion vaccine and a Bcl-2 inhibitor. Other cytokines include, but are not limited to, IL-2, stem cell factor (SCF), IL-3, IL-6, IL-7, IL-12, IL-15, G-CSF, GM-CSF, IL-1α, IL-1β, MIP-1α, LIF, c-kit ligand, TPO, and flt3 ligand.

MUC16 inhibitors can also be used with a DC/tumor fusion vaccine and a Bcl-2 inhibitor to treat cancer. For example, peptide mimetics, such as those described in U.S. Pat. No. 9,633,562, the contents of which are incorporated herein in their entirety, can be used. In some embodiments, the MUC16 peptides and peptide mimetics are used. In some embodiments, the MUC16 peptide contains the four consecutive MUC amino acids CLIC. For example, the peptide can have the amino acid sequence XLIXGVL VTTRRRKK or CLICGVLVTTRRRKK, wherein “X” denotes a cysteine, a penicillamine, homocysteine or 3-mercaptoproline. The peptide can comprise all L amino acids, all D amino acids or a mixture of L and D amino acids. The peptide can be covalently linked to a cell delivery domain or a cell transduction domain. The cell transduction domain can be, for example, an HIV tat cell transduction domain. The cell delivery domain can be, for example, a poly-D-R, poly D-P or poly D-K.

Additional agents that can be used in conjunction with a DC/tumor fusion vaccine and a Bcl-2 inhibitor to treat cancer include TLR agonists, CpG oligodeoxynucleotides (CPG-ODNs), polyinosinic-polycytidylic acid (polyIC), and tetanus toxoid.

Examples of other anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cyclosporin A; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, chlorambucil, fludarabine, dexamethasone (Decadron®), hydrocortisone, methylprednisolone, cilostamide, doxorubicin (Doxil®), forskolin, rituximab, cyclosporin A, cisplatin, vincristine, PDE7 inhibitors such as BRL-50481 and IR-202, dual PDE4/7 inhibitors such as IR-284, cilostazol, meribendan, milrinone, vesnarionone, enoximone and pimobendan, Syk inhibitors such as fostamatinib disodium (R406/R788), R343, R-112 and Excellair® (ZaBeCor Pharmaceuticals, Bala Cynwyd, Pa.). Additional agents include toll-like receptor (TLR) agonist, CpG oligodeoxynucleotides (CPG-ODNs), polyinosinic-polycytidylic acid (polyIC), and tetanus toxoid.

### IV. Methods of Treatment

The present invention provides for both prophylactic and therapeutic methods of treating a subject at risk of (or susceptible to) a cancer. The cancer may be a solid or hematological cancer. In certain embodiments, the cancer is acute myeloid leukemia or multiple myeloma.

a. Prophylactic and Therapeutic Methods

In one aspect, the present invention provides methods for preventing the onset or recurrence of a cancer in a subject by administering to the subject a therapeutically effective amount of a DC/tumor fusion vaccine comprising a dendritic cell fused to a cancer, or tumor, cell and a Bcl-2 inhibitor. In some embodiments, the Bcl-2 inhibitor is a BH3 mimetic. In some embodiments, the Bcl-2 inhibitor is venetoclax. Administration of these prophylactic agents can occur prior to the manifestation of symptoms characteristic of cancer, such that a cancer is prevented or, alternatively, delayed in its progression.

Another aspect of the present invention pertains to methods treating a subject afflicted with cancer, by administering to the subject a therapeutically effective amount of a DC/tumor fusion vaccine comprising a dendritic cell fused to a cancer, or tumor, cell and a Bcl-2 inhibitor. In some embodiments, the Bcl-2 inhibitor is a BH3 mimetic. In some embodiments, the Bcl-2 inhibitor is venetoclax.

Described below are embodiments that pertain to both prophylactic and therapeutic methods of the present invention. Reference to “therapeutically effective amounts” and “therapeutic agent” in the context of a method of treatment refers to therapeutic effective amounts or agents and in the context of a prophylactic treatment, the term refers to prophylactically effective amounts or agents.

As described below and in some embodiments, therapeutically effective amounts of a DC/tumor fusion vaccine comprising a dendritic cell fused to a cancer, or tumor, cell and a Bcl-2 inhibitor are administered to a subject. The DC/tumor fusion vaccine can be used to stimulate the immune system of a mammal for treatment or prevention of cancer. For instance, to treat cancer in a subject, a DC/tumor fusion vaccine containing fused cells formed by the subject's DCs and tumor cells and can be administered to the subject, e.g., at a site near the lymphoid tissue. In some embodiments the subject is in post chemotherapy induced remission. The vaccine can be administered 4 to 12 weeks following the completion of chemotherapy. The vaccine can be administered to four different sites near lymphoid tissue and can be administered multiple times (e.g., two to five) at appropriate intervals (e.g., four weeks) and dosage (e.g., about 10⁵-10⁸, e.g., about 0.5×10⁶ to 1×10⁶, fused cells per administration). Each dose can contain about 1×10⁶ to 1×10⁷ fused cells. As described herein, the DC/tumor fusion vaccine rescues the immune response that results from Bcl-2 inhibitor activity, resulting in a combination therapy that elicits both an immune response and pro-apoptotic activity against a subject's cancer.

The cancer cells will have an immunocompatibility relationship to the subject host and any such relationship is contemplated for use according to the present invention. For example, the cancer cells can be syngeneic. The term “syngeneic” can refer to the state of deriving from, originating in, or being members of the same species that are genetically identical, particularly with respect to antigens or immunological reactions. These include identical twins having matching MHC types. Thus, a “syngeneic transplant” refers to transfer of cells from a donor to a recipient who is genetically identical to the donor or is sufficiently immunologically compatible as to allow for transplantation without an undesired adverse immunogenic response (e.g., such as one that would work against interpretation of immunological screen results described herein).

A syngeneic transplant can be “autologous” if the transferred cells are obtained from and transplanted to the same subject. An “autologous transplant” refers to the harvesting and reinfusion or transplant of a subject's own cells or organs. Exclusive or supplemental use of autologous cells may eliminate or reduce many adverse effects of administration of the cells back to the host, particular graft versus host reaction.

A syngeneic transplant can be “matched allogeneic” if the transferred cells are obtained from and transplanted to different members of the same species yet have sufficiently matched major histocompatibility complex (MHC) antigens to avoid an adverse immunogenic response. Determining the degree of MHC mismatch may be accomplished according to standard tests known and used in the art. For instance, there are at least six major categories of MHC genes in humans, identified as being important in transplant biology. HLA-A, HLA-B, HLA-C encode the HLA class I proteins while HLA-DR, HLA-DQ, and HLA-DP encode the HLA class II proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests may be made of at least 4, and even 5 or 6 MHC antigens within the two or three HLA groups, respectively. In serological MHC tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immunoassays (ELISA). Molecular methods for determining MHC type are well-known and generally employ synthetic probes and/or primers to detect specific gene sequences that encode the HLA protein. Synthetic oligonucleotides may be used as hybridization probes to detect restriction fragment length polymorphisms associated with particular HLA types (Vaughn (2002) Method. Mol. Biol. MHC Protocol. 210:45-60). Alternatively, primers may be used for amplifying the HLA sequences (e.g., by polymerase chain reaction or ligation chain reaction), the products of which may be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method. Mol. Biol. MHC Protocol. 210:67-112).

A syngeneic transplant can be “congenic” if the transferred cells and cells of the subject differ in defined loci, such as a single locus, typically by inbreeding. The term “congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

By contrast, “mismatched allogeneic” refers to deriving from, originating in, or being members of the same species having non-identical major histocompatibility complex (MHC) antigens (i.e., proteins) as typically determined by standard assays used in the art, such as serological or molecular analysis of a defined number of MHC antigens, sufficient to elicit adverse immunogenic responses. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

Similarly, in contrast, “xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

In addition, cancer cells can be obtained from a single source or a plurality of sources (e.g., a single subject or a plurality of subjects). A plurality refers to at least two (e.g., more than one). In still another embodiment, the non-human mammal is a mouse. The animals from which cell types of interest are obtained may be adult, newborn (e.g., less than 48 hours old), immature, or in utero. Cell types of interest may be primary cancer cells, cancer stem cells, established cancer cell lines, immortalized primary cancer cells, and the like. In certain embodiments, the immune systems of host subjects can be engineered or otherwise elected to be immunological compatible with transplanted cancer cells. For example, in one embodiment, the subject may be “humanized” in order to be compatible with human cancer cells. The term “immune-system humanized” refers to an animal, such as a mouse, comprising human HSC lineage cells and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma (null) lack an innate immune system, B cells, T cells, and cytokine signaling), NOG (NOD-SCID IL2r-gamma (truncated)), BRG (BALB/c-Rag2 (null) IL2r-gamma (null)), and H2dRG (Stock-H2d-Rag2 (null) IL2r-gamma (null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988) Science 241:1632-1639, U.S. Pat. No. 7,960,175, and U.S. Pat. Publ. 2006/0161996), as well as related null mutants of immune-related genes like Rag1 (lack B and T cells), Rag2 (lack B and T cells), TCR alpha (lack T cells), perforin (CD8+ T cells lack cytotoxic function), FoxP3 (lack functional CD4+ T regulatory cells), IL2rg, or Prf1, as well as mutants or knockouts of PD-1, PD-L1, Tim3, and/or 2B4, allow for efficient engraftment of human immune cells in and/or provide compartment-specific models of immunocompromised animals like mice (see, for example, PCT Publ. WO2013/062134). In addition, NSG-CD34+ (NOD-SCID IL2r-gamma (null) CD34+) humanized mice are useful for studying human gene and tumor activity in animal models like mice.

As used herein, “obtained” from a biological material source means any conventional method of harvesting or partitioning a source of biological material from a donor. For example, biological material may obtained from a solid tumor, a blood sample, such as a peripheral or cord blood sample, or harvested from another body fluid, such as bone marrow or amniotic fluid. Methods for obtaining such samples are well-known to the artisan. In the present invention, the samples may be fresh (i.e., obtained from a donor without freezing). Moreover, the samples may be further manipulated to remove extraneous or unwanted components prior to expansion. The samples may also be obtained from a preserved stock. For example, in the case of cell lines or fluids, such as peripheral or cord blood, the samples may be withdrawn from a cryogenically or otherwise preserved bank of such cell lines or fluid. Such samples may be obtained from any suitable donor.

The obtained populations of cells may be used directly or frozen for use at a later date. A variety of mediums and protocols for cryopreservation are known in the art. Generally, the freezing medium will comprise DMSO from about 5-10%, 10-90% serum albumin, and 50-90% culture medium. Other additives useful for preserving cells include, by way of example and not limitation, disaccharides such as trehalose (Scheinkonig et al. (2004) Bone Marrow Transplant. 34:531-536), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% dimethyl sulfoxide (DMSO), and 2% hetastarch. Other compositions and methods for cryopreservation are well-known and described in the art (see, e.g., Broxmeyer et al. (2003) Proc. Natl. Acad. Sci. U.S.A. 100:645-650). Cells are preserved at a final temperature of less than about −135° C.

c. Combination Therapy

The DC/tumor fusion vaccine can be administered in combination therapy with additional therapeutic agents including, but not limited to, additional Bcl-2 inhibitors, hypomethylation (HMA) agents, checkpoint inhibitor therapy (e.g., a PD-1, PDL1, PDL2, TIM3, LAG3, or RGMb inhibitor), GM-CSF, an immunomodulator (e.g., lenalidomide, pomalinomide, or apremilast), chemotherapeutic agents, hormones, antiangiogens, radiolabelled compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. The additional therapeutic agent is administered contemporaneously with the DC/tumor fusion vaccine, prior to administration of the DC/tumor fusion vaccine, or after administration of the DC/tumor fusion vaccine. For example, the additional therapeutic agent can be administered 1 week prior to or after the DC/tumor fusion vaccine. In some embodiments, the additional therapeutic agent is administered at 1, 2, 3, 4, 5, or 6 week intervals.

Thus, in certain embodiments, agents disclosed herein may be used alone or conjointly administered with another type of therapeutic agent. For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In certain embodiments, provided herein are methods comprising administering to a subject a DC/tumor fusion vaccine and/or a Bcl-2 inhibitor at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, the DC/tumor fusion vaccine is administered in combination with a Bcl-2 inhibitor. The Bcl-2 inhibitor acts to suppress anti-apoptotic effects caused by Bcl-2 activity. The Bcl-2 can be a BH3 mimetic such as venetoclax. In some embodiments, the Bcl-2 inhibitor is an antisense oligonucleotide. Other Bcl-2 inhibitors are contemplated as well, including those undergoing clinical trials or in preclinical development, such as BGB-11417, G3139, and oblimersen.

In certain embodiments, provided herein is a composition, e.g., a pharmaceutical composition, containing at least one agent described herein together with a pharmaceutically acceptable carrier. In one embodiment, the composition includes a combination of multiple (e.g., two or more, three or more, four or more, or five or more) agents described herein.

In some embodiments, the DC/tumor fusion vaccine is administered in combination with at least one hypomethylation agent (HMA). The HMA acts to reduce or inhibit methylation of DNA in a cell, such as a cancer cell. Hypermethylation of CpG islands in the promoters of tumor suppressor genes results in decreased or silenced expression of these genes. Demethylation of these CpG islands allows for the expression of these tumor suppression genes. Other epigenetic effects elicited by hypermethylation can be mitigated or eliminated by HMA activity. The HMA can be approved for clinical use, undergoing clinical trials, or in preclinical development (e.g., decitabine, 5-azacytidine, guadecitabine, and 5-fluro-2′-deoxycytidine).

In one embodiment, the DC/tumor fusion vaccine can be administered with a therapeutically effective dose of chemotherapeutic agent. In another embodiment, the DC/tumor fusion vaccine is administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

The DC/tumor fusion vaccine and Bcl-2 combination therapy can also be administered in combination with targeted therapy, e.g., immunotherapy. The term “targeted therapy” refers to administration of agents that selectively interact with a chosen biomolecule to thereby treat cancer. For example, targeted therapy regarding the inhibition of immune checkpoint inhibitor is useful in combination with the methods of the present invention. The term “immune checkpoint inhibitor” means a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD39, CD73, CD48, 2B4 (CD244), B7.1, B7.2, ILT-2, ILT-4, TIGIT, RGMb, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer vaccine is administered in combination with one or more inhibitors of immune checkpoints, such as PD1, PD-L1, and/or CD47 inhibitors. Checkpoint inhibitors can be administered at intervals, for example, at 4 to 6 week intervals.

Immunotherapy is one form of targeted therapy that may comprise, for example, the use of additional cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used in conjunction with the DC/tumor fusion vaccine and Bcl-2 inhibitor combination therapy. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. The foregoing examples of chemotherapeutic agents are illustrative, and are not intended to be limiting.

In some embodiments, in addition to the DC/tumor fusion vaccine, the subject further receives granulocyte-macrophage colony-stimulating factor (GM-CSF). GM-CSF can be administered on the day the cell fusions are administered. In some embodiments, GM-CSF is administered daily for a period of time (e.g., 3, 4, or 5 or more subsequent days). The GM-CSF can be administered subcutaneously at a dose of 100 μg. The GM-CSF can be administered at the site where the cell fusions are administered.

In some embodiments, a DC/tumor fusion vaccine and a Bcl-2 inhibitor can be administered to a subject in conjunction with a MUC-1 inhibitor, such as GO-203 to treat a cancer.

An inhibitor of indoleamine 2,3-dioxygenase (IDO) can be administered to a subject in conjuction with a DC/tumor fusion vaccine and a Bcl-2 inhibitor to treat cancer. In some embodiments, the IDO inhibitor is INB024360 or 1-MDT.

In some embodiments, an immunomodulatory agent can be administered in conjunction with a DC/tumor fusion vaccine and a Bcl-2 inhibitor to treat cancer. Examples of immunomodulatory agents include, but are not limited to, lenalidomide, pomalinomide, and apremilast.

MUC16 inhibitors can also be administered with a DC/tumor fusion vaccine and a Bcl-2 inhibitor to treat cancer. In some embodiments, the MUC16 peptide contains the four consecutive MUC amino acids CLIC. For example, the peptide can have the amino acid sequence XLIXGVLVTTRRRKK or CLICGVLVTTRRRKK, wherein “X” denotes a cysteine, a penicillamine, homocysteine or 3-mercaptoproline. Suitable dosages of a MUC16 inhibitor are in the range of 0.0001 mg/kg-100 mg/kg and can be administered daily.

Additional agents that can be administered in conjunction with a DC/tumor fusion vaccine and a Bcl-2 inhibitor to treat cancer include TLR agonists, CpG oligodeoxynucleotides (CPG-ODNs), polyinosinic-polycytidylic acid (polyIC), and tetanus toxoid.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Subjects are advised to avoid direct sunlight and bright indoor light for at least 6 weeks. If subjects must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in subjects for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO2) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO2 laser is also able to cut the skin. The laser is used in this way to remove skin cancers.

Neodymium: yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outsubject basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO2 and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a subject's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The immunotherapy and/or cancer therapy may be administered before, after, or concurrently with the cancer vaccine and Bcl-2 inhibitor described herein. The duration and/or dose of treatment with the cancer vaccine may vary according to the particular cancer vaccine, or the particular combinatory therapy. An appropriate treatment time for a particular cancer therapeutic agent will be appreciated by the skilled artisan. The present disclosure contemplates the continued assessment of optimal treatment schedules for each cancer therapeutic agent, where the phenotype of the cancer of the subject as determined by the methods encompassed by the present invention is a factor in determining optimal treatment doses and schedules.

### V. Clinical Efficacy

Clinical efficacy can be measured by any method known in the art. For example, the response to a cancer therapy (e.g., a DC/tumor fusion vaccine and anti-Bcl-2 combination, or a DC/tumor fusion vaccine, anti-Bcl-2, and hypomethylation agent combination), relates to any response to the cancer to the therapy. Clinical efficacy in hematological cancers can be assessed by detecting differences in cell count or maturity blood cells before and after treatment, for example, by using microscopy or fluorescence assisted cell sorting (FACS) analysis. Tumor response, for example, may be assessed by comparing the size of a tumor after treatment to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of cancer or tumor response may be performed after the onset of treatment, e.g., after a few hours, days, weeks or preferably after a few months. A typical endpoint for response assessment is upon termination of therapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of subjects who are in complete remission (CR), the number of subjects who are in partial remission (PR) and the number of subjects having stable disease (SD) at a time point at least 6 months out from the end of therapy. The shorthand for this formula is CBR=CR+PR+SD over 6 months. In some embodiments, the CBR for a particular cancer vaccine therapeutic regimen is at least 25%, 30%, 35%, 40%, 45%, 50%, 55%, 60%, 65%, 70%, 75%, 80%, 85%, or more.

Additional criteria for evaluating the response to cancer therapy (e.g., a DC-tumor fusion vaccine and a Bcl-2 inhibitor) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to therapy, probability of survival, probability of metastasis within a given time period, and probability of tumor recurrence.

For example, in order to determine appropriate threshold values, a particular agent encompassed by the present invention can be administered to a population of subjects and the outcome can be correlated to biomarker measurements that were determined prior to administration of any cancer therapy (e.g., a DC-tumor fusion vaccine and a Bcl-2 inhibitor). The outcome measurement may be pathologic response to therapy. Alternatively, outcome measures, such as overall survival and disease-free survival can be monitored over a period of time for subjects following cancer therapy (e.g., a DC-tumor fusion vaccine and a Bcl-2 inhibitor) for whom biomarker measurement values are known. In certain embodiments, the same doses of the agent are administered to each subject. In related embodiments, the doses administered are standard doses known in the art for the agent. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy (e.g., a DC-tumor fusion vaccine and a Bcl-2 inhibitor) can be determined using methods such as those described in the Examples section.

### VI. Pharmaceutical Compositions and Administration

For DC-tumor fusion vaccines of present invention, DC-tumor fusion cells can be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective.

The DC-tumor fusion vaccine of the present invention can be administered in any suitable route as described herein, such as by infusion. DC-tumor fusion vaccines can also be administered before, concurrently with, or after, other anti-cancer agents (e.g., a Bcl-2 inhibitor, such as venetoclax).

Administration can be accomplished using methods generally known in the art.

Agents, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine of the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. (2005) Blood 106:1565-1573; Shultz et al. (2005) J. Immunol. 174:6477-6489; and Holyoake et al. (1999) Exp. Hematol. 27:1418-1427).

Two or more cell types can be combined and administered, such as DC-tumor fusion cells and adoptive cell transfer of stem cells, DC-tumor fusion vaccine cells and other cell-based vaccines, and the like. For example adoptive cell-based immunotherapies can be combined with the DC-tumor fusion vaccine of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like. The ratio of cancer cells in the cancer vaccine described herein to other cell types can be 1:1, but can modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or can signal the time for tumor harvesting. Any such metrics are variables that can be adjusted according to well-known parameters in order to determine the effect of the variable on a response to anti-cancer immunotherapy. In addition, the transplanted cells can be co-transplanted with other agents, such as cytokines, extracellular matrices, cell culture supports, and the like.

The DC/tumor fusion vaccine can be used to stimulate the immune system of a mammal for treatment or prevention of cancer.

In addition, anti-cancer agents (e.g., DC-tumor fusion vaccine, Bcl-2 inhibitors, hypomethylation inhibitors, cytokines, IDO inhibitors, checkpoint inhibitors, immunomodulatory agents, TLR agonists, polyIC, CPG-ODN, MUC1 inhibitors, and MUC16 inhibitors, and the like) of the present invention can be administered to subjects or otherwise applied outside of a subject body in a biologically compatible form suitable for pharmaceutical administration. By “biologically compatible form suitable for administration in vivo” is meant a form to be administered in which any toxic effects are outweighed by the therapeutic effects. Administration of an anti-cancer agent as described herein can be in any pharmacological form including a therapeutically active amount of an agent alone or in combination with a pharmaceutically acceptable carrier. The phrase “therapeutically-effective amount” as used herein means that amount of an agent that is effective for producing some desired therapeutic effect, e.g., cancer treatment, at a reasonable benefit/risk ratio.

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

A combination dosage form or simultaneous administration of single agents can result in effective amounts of each desired modulatory agent present in the subject at the same time.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The agent may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent encompassed by the present invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms encompassed by the present invention are dictated by, and directly dependent on, (a) the unique characteristics of the active compound and the particular therapeutic effect to be achieved, and (b) the limitations inherent in the art of compounding such an active compound for the treatment of sensitivity in individuals.

Compositions of the present invention can be administered one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays

### VII. Kits

The present invention also encompasses kits. For example, the kit can comprise DC/tumor fusion vaccines, Bcl-2 inhibitors, hypomethylation agents, immune checkpoint inhibitors, and combinations thereof, packaged in a suitable container and can further comprise instructions for using such reagents. The kit may also contain other components, such as administration tools packaged in a separate container.

Other embodiments of the present invention are described in the following Examples. The present invention is further illustrated by the following examples which should not be construed as further limiting. The contents of all references, patents and published patent applications cited throughout this application, as well as the Figures, are incorporated herein by reference.

### EXEMPLIFICATION

This invention is further illustrated by the following examples, which should not be construed as limiting.

### Example 1: Treating Cancer Using a DC/Tumor Fusion Vaccine and a Bcl-2 Inhibitor

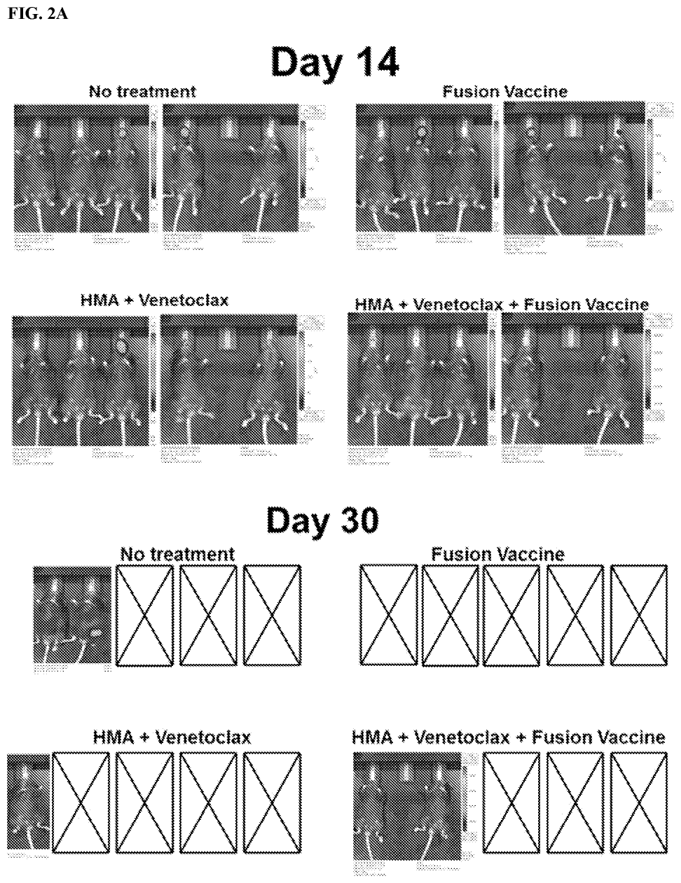

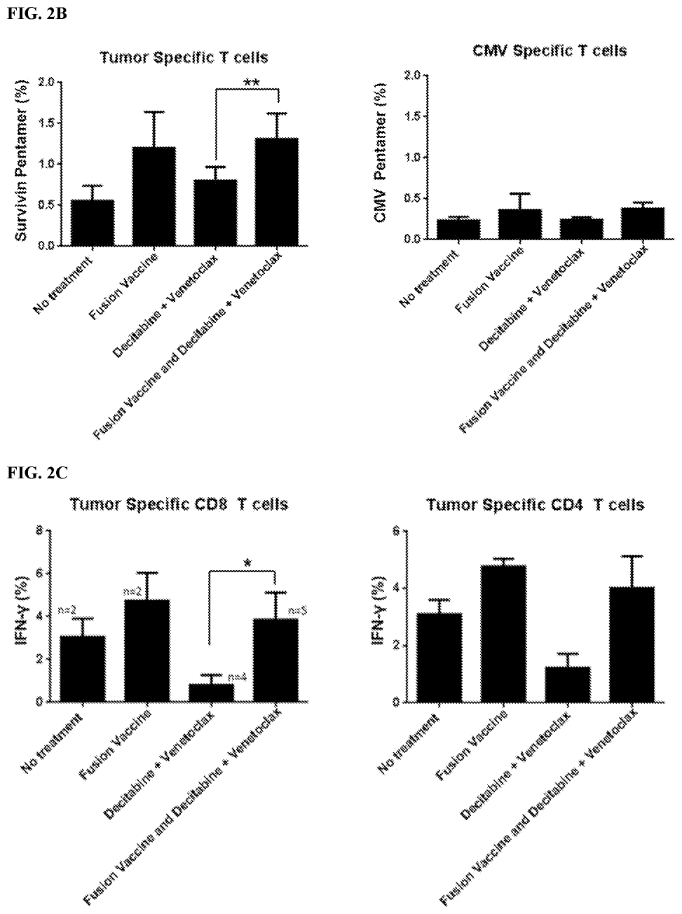

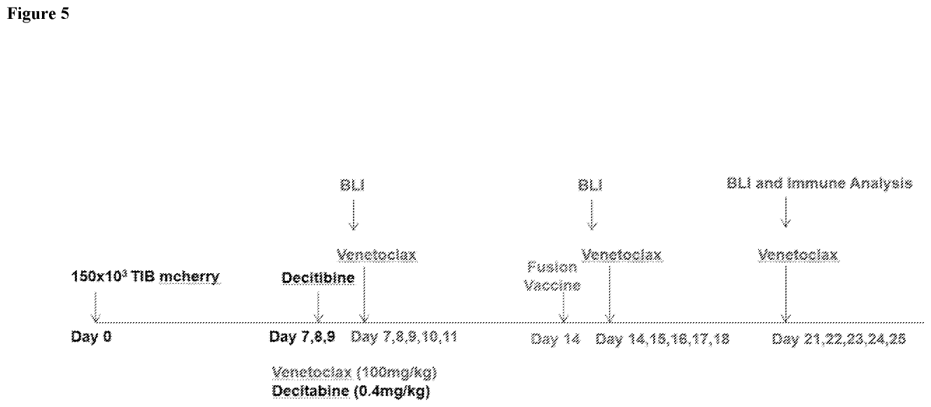

AML model mice were used as test subjects of a DC/Tumor fusion vaccine, venetoclax, and decitabine combination therapy. The subjects were divided into four test groups which received 1) no treatment, 2) a DC/tumor fusion vaccine, 3) decitabine and venetoclax, or 4) a DC/tumor fusion vaccine, decitabine and venetoclax. Bioluminescence imaging was performed at 14 and 30 days post administration. Each mouse was alive at day 14 (FIG. 2A). However, at day 30 none of the five mice that received only the DC/tumor fusion vaccine and one of the five mice that received venetoclax and decitabine survived. At the 30-day time point, two of the five mice that received both the venetoclax and decitabine survived (FIG. 2B). Exemplary in vivo treatment schema with fusion vaccine and decitabine+venetoclax is shown in FIG. 5.

The combination of venetoclax and decitabine would be expected to inhibit an immune response as venetoclax suppress blood cell counts. To determine if this combination muted the immune response that was intended to be elicited by administering the DC/tumor fusion vaccine, tumor specific T cells were isolated from each test subject. These T cells were then evaluated for the presence of survivin-specific T cells to determine the robustness of the immune response generated against the cancer. Referring to FIG. 2A, mice that were administered only decitabine and venetoclax had a muted response relative to that observed in the mice that received only the DC/tumor fusion vaccine or the DC/tumor fusion vaccine in combination with decitabine and venetoclax. These data indicate that the DC/tumor fusion vaccine rescues the immune response that is otherwise muted by decitabine and venetoclax.

To confirm that the treatment regimen resulted in a tumor specific immune response, the mice were evaluated for the presence of cytomegalovirus (CMV)-specific T cells. As shown in FIG. 2B, none of the 4 groups of mice generated a substantial number of CMV-specific T cells, indicating that the immune response generated in the treatment groups receiving the DC/tumor fusion vaccine was tumor specific.

The expression of IFN-γ by T cells is a measure of the cells' activity. Tumor specific T cells from each treatment group were further evaluated for activity levels. As shown in FIGS. 2C and 2D, the mice receiving only the DC/tumor fusion vaccine generated tumor specific CD4 and CD8 T cells that exhibited the most activity. Further, for mice that were administered the DC/tumor fusion vaccine, decitabine, and venetoclax, administration of the DC/tumor fusion vaccine rescued the CD4 and CD8 T cell activity that is muted in the mice that were administered only decitabine and venetoclax.

### Example 2: Hypomethylating Agents Augment Tumor Antigen Presentation

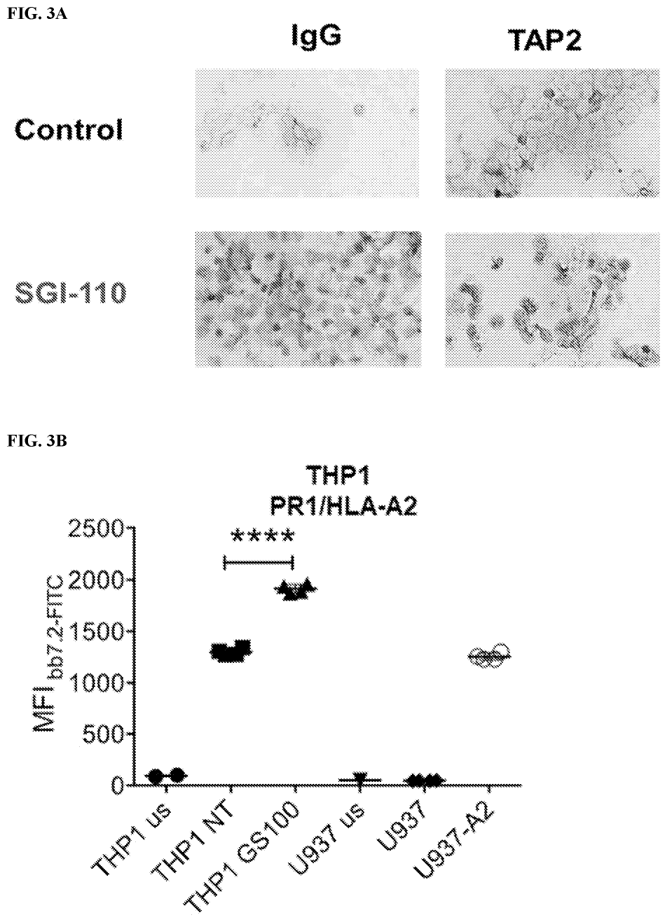

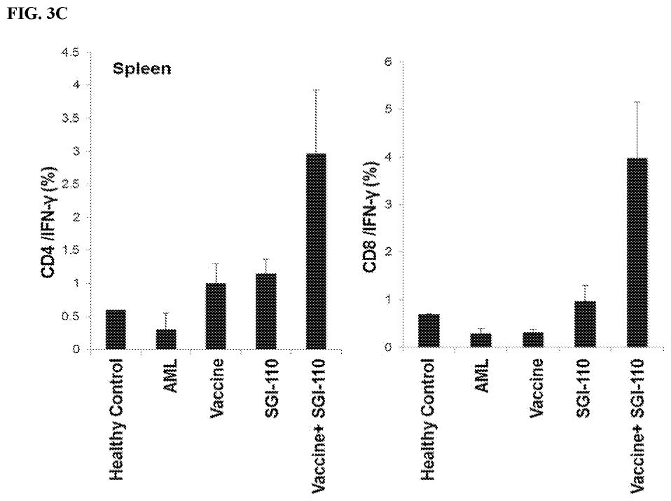

To determine the effect hypomethylation agents (HMAa) have on tumor antigen presentation, AML patient-derived tumor cells were treated for two days with four doses of 1 μM SGI-110, an HMA, added twice daily and then cultured for additional two days. TAP2 expression was assessed using immunohistochemical staining with and without treatment with SGI-110 (FIG. 3A). The HLA-A2 expressing AML cell line, THP-1, was treated for two days with four doses of 1 μM SGI-110 added twice daily and then cultured for an additional two days. The cells were then analyzed for binding capacity of a unique T cell receptor-like (TCRL) antibody. The TCRL antibody is a TCR mimic that recognizes the PR1-HLA-A2-MHC complex on the surface of leukemic cells where PR-1 is a known leukemic antigen. Following exposure of SGI-110 to THP-1, enhanced tumor antigen presentation as evidenced by increased binding of TCRL antibody observed via flow cytometric analysis (FIG. 3B). An HLA-A2 negative cell line U937 (histiocytic lymphoma) was used as a negative control.

Treatment with HMA with fusion vaccination enhances leukemia-specific immunity and survival in murine syngeneic AML model. C57BL/6J mice were retro-orbitally inoculated with 100,000 syngeneic TIB-49 AML cells that were stably transduced with GFP. DC/AML fusion cells were generated. The mice were then treated with either vehicle control, SGI-110 (1 mg/kg)×5 days, vaccine alone, or combination of SGI-110 and the fusion vaccine. The mice were followed for survival and splenocytes were harvested. The cells were then exposed ex vivo to autologous tumor lysate for 5 days. The cells underwent intracellular flow cytometric analysis for IFN-γ expression in both CD4+ and CD8+ T cells. Therapy with SGI-110 plus the vaccine shows a significant increase in tumor-specific T cells as detected by IFN-γ production in CD4 and CD8 splenocytes as compared to treatment with the fusion vaccine or SGI-110 monotherapy.

### Example 3: DC/Tumor Fusion Vaccines Combined with Checkpoint Inhibits Prevent Establishment of AML

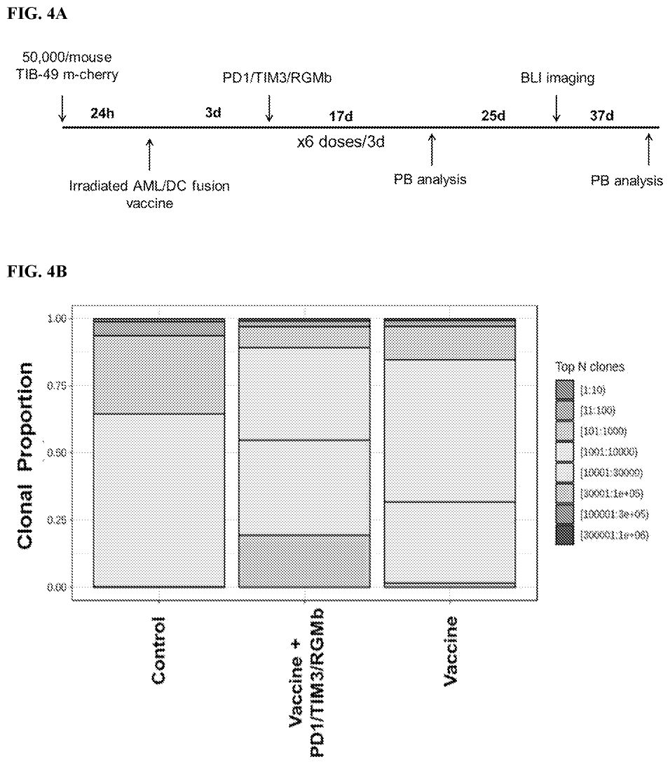

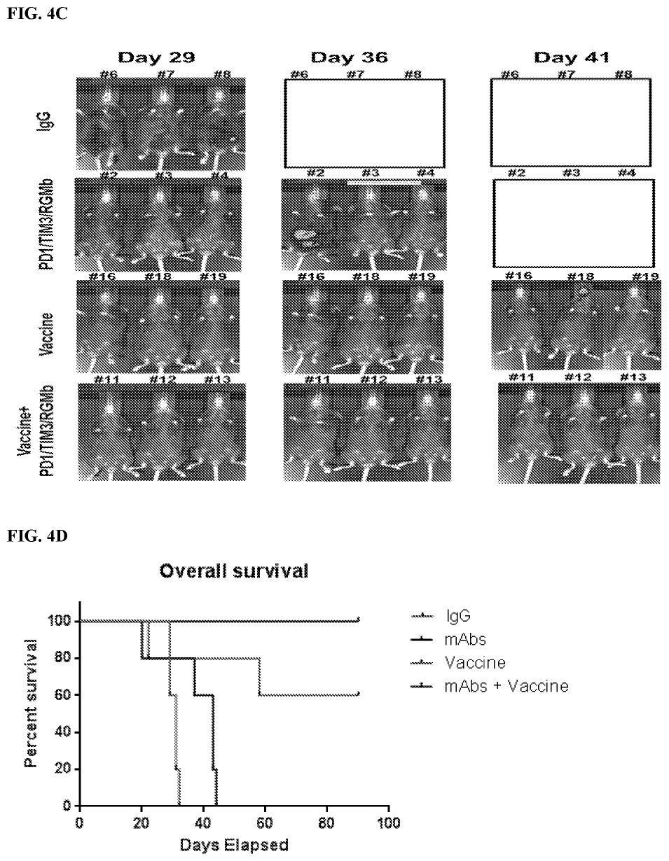

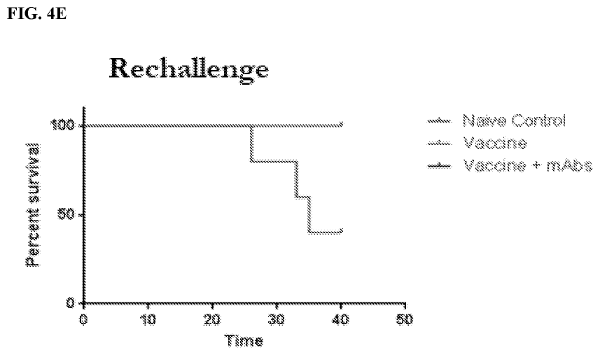

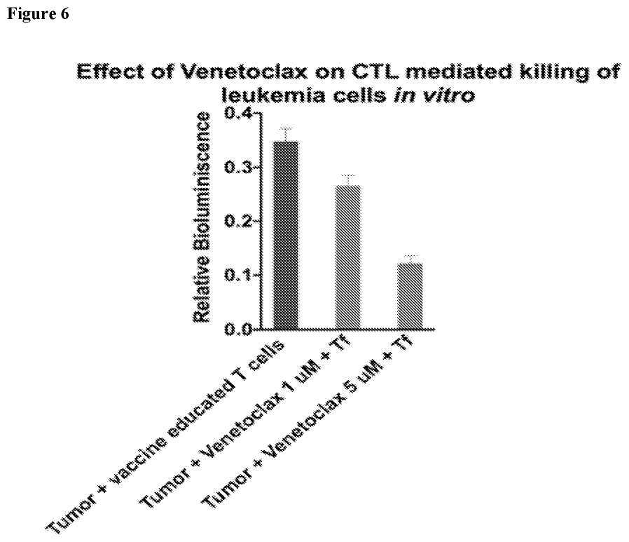

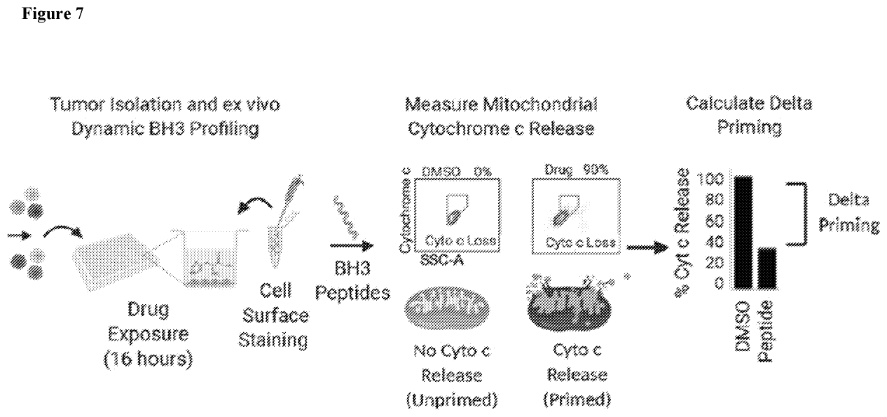

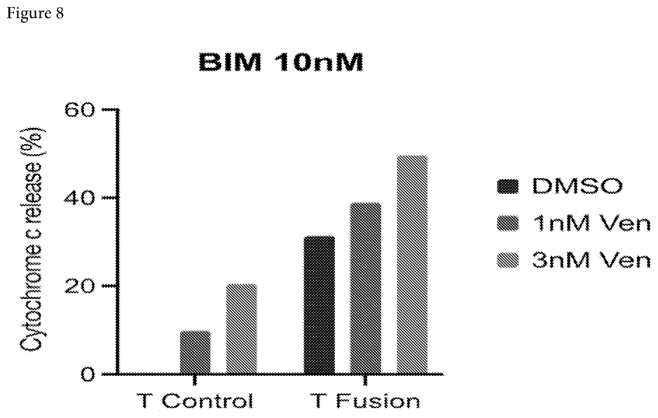

To determine if a combination treatment with a DC/AML fusion vaccine and checkpoint inhibitor therapy prevents establishment of AML in vivo. C57BL/6J mice were retro-orbitally inoculated with 50×10³ syngeneic TIB-49 AML cells that were stably transduced with luciferase/m-cherry. Syngeneic DC/AML fusion cells were generated and evaluated for co-expression of tumor (m-cherry) and DC (CD86) markers using flow cytometry. The mice were then treated with either vaccine alone, anti-PD1/TIM3/RGMb antibodies, or combination of anti-PD1/TIM3/RGMb antibodies and the fusion vaccine according to the timeline shown in FIG. 4A. The mice were treated with appropriate isotype control as a negative control. Vaccination followed by the checkpoint blockade resulted in upregulation of genes regulating activation and proliferation of memory and effector T cells as well as enhanced T cell clonal diversity (FIG. 4B). Bioluminescence imaging was performed serially starting on day 29 post-inoculation (three representative mice are shown) (FIG. 4C) and the mice were followed for survival for 90 days as demonstrated in a Kaplan Meier curve (FIG. 4D). Mice were rechallenged and followed for survival (FIG. 4E). These findings indicate that sequential expansion of vaccine educated lymphocytes followed by checkpoint blockade selectively promote vaccine-mediated tumor specific immune responses. Results shown in FIG. 6-8 further support venetoclax and vaccine-mediated impact on tumor cell lysis.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

1. A method of treating a cancer in a subject, the method comprising administering to the subject therapeutically effective amounts of a dendritic cell (DC)/tumor fusion vaccine and a Bcl-2 inhibitor.

2-3. (canceled)

4. The method of claim 1, further comprising administering to the subject a hypomethylation agent.

5. The method of claim 1, wherein the cancer is a solid tumor or hematological cancer.

6. The method of claim 5, wherein the hematological cancer is acute myeloid leukemia (AML), multiple myeloma (MM), or chronic lymphocytic leukemia (CLL).

7. The method of any one of claims 1-3, wherein the Bcl-2 inhibitor is a BH3 mimetic.

8. The method of claim 7, wherein the Bcl-2 mimetic is venetoclax.

9. The method of claim 4, wherein the hypomethylation agent is decitabine, 5-azacytidine, guadecitabine, or 5-fluro-2′-deoxycytidine.

10. The method of claim 1, wherein the DC/tumor fusion vaccine is autogenic.

11. The method of claim 1, wherein the DC/tumor fusion vaccine is allogenic.

12. The method of claim 1, further comprising administering to the subject an immune checkpoint inhibitor.

13. The method of claim 12, wherein the checkpoint inhibitor is selected from the group of a PD-1 inhibitor, a TIM-3 inhibitor, a LAG3 inhibitor, a TIGIT inhibitor, a B7H3 inhibitor, a CD39 inhibitor, a CD73 inhibitor, and an adenosine A2A receptor.

14. (canceled)

15. The method of claim 1, further comprising administering to the subject an immunomodulatory agent.

16. The method of claim 15, wherein the immunomodulatory agent is lenalidomide or pomalidomide.

17. The method of claim 1, further comprising administering to the subject a cytokine.

18. The method of claim 17, wherein the cytokine is granulocyte-macrophage colony-stimulating factor (GM-CSF).

19. The method of claim 1, further comprising administering an IDO inhibitor.

20. The method of claim 1, further comprising administering a toll-like receptor (TLR) agonist, CpG oligodeoxynucleotides (CPG-ODNs), polyinosinic-polycytidylic acid (polyIC), or tetanus toxoid.

21. The method of claim 1, further comprising administering a MUC1 inhibitor.

22. The method of claim 21, wherein the MUC1 inhibitor is GO-203.

23. (canceled)

24. The method of claim 1, wherein the subject is in remission.

25. The method of claim 1, wherein the subject has minimal residual disease.

26. The method of claim 4, wherein the hypomethylation agent is further administered as a maintenance treatment.


SEQUENCE LISTING (4 sequences)

SEQ ID NO: 1 - DNA, 6881 bp, Artificial Sequence
  synthetic construct
acccctcgccgcaccacacacagcgcgggcttctagcgctcggcaccggcgggccaggcgcgtcctgccttcatttatccagcagcttttcggaaaatgcatttgctgttcggagtttaatcagaagaggattcctgcctccgtccccggctccttcatcgtcccctctcccctgtctctctcctggggaggcgtgaagcggtcccgtggatagagattcatgcctgtgcccgcgcgtgtgtgcgcgcgtgtaaattgccgagaaggggaaaacatcacaggacttctgcgaataccggactgaaaattgtaattcatctgccgccgccgctgccttttttttttctcgagctcttgagatctccggttgggattcctgcggattgacatttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 2 - protein, 239 aa, Artificial Sequence
  synthetic construct
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - DNA, 1595 bp, Artificial Sequence
  synthetic construct
acccctcgccgcaccacacacagcgcgggcttctagcgctcggcaccggcgggccaggcgcgtcctgccttcatttatccagcagcttttcggaaaatgcatttgctgttcggagtttaatcagaagaggattcctgcctccgtccccggctccttcatcgtcccctctcccctgtctctctcctggggaggcgtgaagcggtcccgtggatagagattcatgcctgtgcccgcgcgtgtgtgcgcgcgtgtaaattgccgagaaggggaaaacatcacaggacttctgcgaataccggactgaaaattgtaattcatctgccgccgccgctgccttttttttttctcgagctcttgagatctccggttgggattcctgcggattgacatttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaggccacaggtccgagatgcgggggttggagtgcgggtgggctcctggggcaatgggaggctgtggagccggcgaaataaaatcagagttgttgct

SEQ ID NO: 4 - protein, 205 aa, Artificial Sequence
  synthetic construct
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG